(19) United States
(12) Patent Application Publication
(10) Pub. No.: US 2026/0290537 A1
(43) Pub. Date: Sep. 24, 2026

(54) MEDICATION DELIVERY DEVICE CONNECTION TECHNIQUES

(71) Applicant: Eli Lilly and Company, Indianapolis, IN (US)

(72) Inventors: Rachel Lauren MORGAN, Harvard, MA (US); Samiullah Khan DURRANI, Harvard, MA (US); Jennal Lynn JOHNSON, Chicago, IL (US); Patrick Kristian-Collin LIND, Westfield, IN (US); Kailee Nichole QUEENER, Indianapolis, IN (US); Jason Joseph SHEREY, Poland, IN (US); Isaac Andrew SMITH, Avon, IN (US)

(21) Appl. No.: 19/480,255

(22) PCT Filed: May 9, 2024
(86) PCT No.: PCT/US2024/028552

§ 371 Date: Oct. 30, 2025

Related U.S. Application Data
(60) Provisional application No. 63/501,822, filed on May 12, 2023.

(51) Int. Cl. G16H 20/17; A61M 5/315; G16H 40/67

(52) U.S. Cl. CPC G16H 20/17; A61M 5/31568; G16H 40/67; A61M 2205/3584; A61M 2205/502; A61M 2205/6072

(57) ABSTRACT

A method is provided for presenting information from a particular medication delivery device to a user. The method includes: receiving a set of advertising packets, the set of advertising packets comprising at least one advertising packet from one or more medication delivery devices, the one or more medication delivery devices including the particular medication delivery device, wherein each advertising packet includes an identifier associated with a respective medication delivery device of the one or more medication delivery devices; determining a number of unique identifiers included in the set of advertising packets to determine a number of transmitting medication delivery devices; determining, based on the number of transmitting medication delivery devices, whether to prompt the user to provide identification information for the particular medication delivery device; and presenting the information from the particular medication delivery device to the user.

### FIELD OF THE INVENTION

The techniques described herein relate generally to medication delivery device connection techniques, including medication delivery device connection protocols, status inquiries for medication delivery devices, retiring medication delivery devices, and managing user accounts for medication delivery devices.

### BACKGROUND OF THE INVENTION

Patients suffering from various diseases must frequently inject themselves with medication. To allow a person to conveniently and accurately self-administer medicine, a variety of devices broadly known as pen injectors or injection pens have been developed. Generally, these pens are equipped with a cartridge including a piston and containing multi-dose quantity of liquid medication. A drive member is moveable forward to advance the piston in the cartridge to dispense the contained medication from an outlet at the distal cartridge end, typically through a needle.

Some injector pens include a functionality to communicate with another device (e.g., smartphone). For example, a handshake or pairing procedure may be performed to establish a wireless communication link or session between the injector pen and device.

### SUMMARY OF THE INVENTION

According to an exemplary embodiment of the present disclosure, a method is provided for presenting information from a particular medication delivery device to a user. The method includes: receiving a set of advertising packets, the set of advertising packets comprising at least one advertising packet from one or more medication delivery devices, the one or more medication delivery devices including the particular medication delivery device, wherein each advertising packet includes an identifier associated with a respective medication delivery device of the one or more medication delivery devices; determining a number of unique identifiers included in the set of advertising packets to determine a number of transmitting medication delivery devices; determining, based on the number of transmitting medication delivery devices, whether to prompt the user to provide identification information for the particular medication delivery device; and presenting the information from the particular medication delivery device to the user.

According to another exemplary embodiment of the present disclosure, a method is provided for wirelessly connecting to a medication delivery device. The method includes: receiving a device identifier associated with the medication delivery device at a computing device associated with a user; determining, based on the device identifier, whether the medication delivery device is associated with a different user other than the user; upon determining that the medication delivery device is not associated with the different user, storing, at the computing device, information associated with the medication delivery device, the information comprising an encryption key; receiving, at the computing device, encrypted information from the medication delivery device; and using the encryption key to decrypt the encrypted information received from the medication delivery device.

According to another exemplary embodiment of the present disclosure, a method is provided for prompting a user of a medication delivery device to connect the device to a user account of the user. The method includes: receiving, at a computing device, a message from the medication delivery device, the message comprising: identification information for the medication delivery device; and dose information indicative of a number of doses administered by the medication delivery device; determining, based on the identification information of the medication delivery device, whether the medication delivery device is associated with any user account; upon determining that the medication delivery device is not associated with any user account, and when the number of doses administered satisfies error message criteria, displaying an error message at the computing device indicating that the medication delivery device should be associated with the user account of the user, without preventing the user from using the medication delivery device prior to associating the medication delivery device with any user account.

According to another exemplary embodiment of the present disclosure, a method for retiring a medication delivery device. The method includes: accessing data associated with a medication delivery device, wherein: the medication delivery device is in a list of active medication delivery devices associated with a user account; and the medication delivery device comprises: a reservoir sized sufficiently to hold medication for a plurality of doses; and a wireless communication unit that wirelessly transmits information about the medication delivery device; determining, based on the accessed data, to retire the medication delivery device; and retiring the medication delivery device, wherein retiring the medication delivery device comprises at least one of: removing the medication delivery device from the list of medication delivery devices associated with the user account; and (b) moving an entry for the medication delivery device from the list of active medication delivery devices associated with the user account to a list of retired medication delivery devices.

### BRIEF DESCRIPTION OF THE DRAWINGS

Additional embodiments of this disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a perspective view of a medication delivery device having a dose detection system according to aspects of the present disclosure.

FIG. 2 is a partially exploded perspective view of the medication delivery device of FIG. 1, showing a dose button having a support and a cover, where the cover is shown separated from the support.

FIG. 3 is a partially exploded perspective view of the medication delivery device of FIG. 1 showing the components of the dose detection system.

FIG. 4 is a cross-sectional view of the medication delivery device of FIG. 1.

FIG. 5 is a partial cutaway view of a proximal end of the medication delivery device of FIG. 1, showing components of the dose detection system.

FIG. 6 is an underside view of a portion of the dose button of FIG. 1, showing a printed circuit board held within the dose button cover.

FIG. 7 is an exploded view of the portion of the dose button shown in FIG. 6.

FIG. 8 is a perspective view of a flange of a dose detection system of a medication delivery device.

FIG. 9 is a top down view of the flange of FIG. 8.

FIG. 10 is a perspective view of a dose button support.

FIG. 11 is a top down view of the dose button support of FIG. 10.

FIG. 12 is a diagram of an example system for wirelessly communicating with a medication delivery device, according to some embodiments.

FIG. 13 is a flowchart showing an exemplary method for wirelessly connecting to a medication delivery device, according to some embodiments.

FIG. 14 is a flowchart showing an exemplary method for transitioning a medication delivery device from an unconnected to a connected state.

FIG. 15 is a flowchart of an exemplary method for processing an advertising packet received from a particular medication delivery device, according to some embodiments.

FIG. 16 is a flowchart of an exemplary method for presenting information in response to a query for information on a particular medication delivery device, according to some embodiments.

FIG. 17 is a flowchart of an exemplary method for determining whether to retire a medication delivery device, according to some embodiments.

FIG. 18 is a flowchart of an exemplary method for retiring a medication delivery device that has little or no medicine, a low battery, and/or is otherwise unusable, according to some embodiments.

FIG. 19 is a flowchart showing an exemplary method for preventing communications with a medication delivery device subject to recall, according to some embodiments.

### DETAILED DESCRIPTION OF THE INVENTION

For the purpose of promoting an understanding of the principles of the present disclosure, reference will now be made to the embodiments illustrated in the drawings, and specific language will be used to describe the same. It will nevertheless be understood that no limitation of the scope of the invention is thereby intended.

The present disclosure relates to software-based techniques for connecting to and processing information received from the medication delivery device. According to some embodiments, the techniques can receive and process information about a status of the medication delivery device and/or information about a medication that was delivered to a patient, such as a time of an injection event, a date of an injection event, a dose amount of the injection event, and/or other injection or dose-related data. For example, the techniques can present such information to a patient, which may help the patient to track dosing information over time. Such information can be used, for example, to plan dosing schedules, such as to determine when to take their next dose of medication, which type of medication to take for their next dose, and/or how much of a medication to take for their next dose. Such dosing information can also be used by the patient's health care provider, payer, researcher conducting a clinical trial in which the patient is taking part, or other interested entity, to track patient's adherence to medication regimens over time.

According to some embodiments, the techniques can receive the information in response to a query transmitted to the medication delivery device and/or through wireless packets, such as advertising packets, broadcast by the medication delivery device. Protocols for wirelessly communicating data between a transmitting device and a receiving device often require performing a handshake or pairing procedure between the transmitting and receiving devices to establish a wireless communication link or session therebetween. However, as used herein, the term “advertising packets” is used to refer to wireless transmissions used by a transmitting device (e.g., a medication delivery device) to convey data to a receiving device (e.g., a computing device, such as a smartphone) without requiring any handshake or pairing process between the transmitting and receiving device. The inventors have appreciated that, by using advertising packets the user may not need to pair the medication delivery device and the computing device in order to process information from a medication delivery device. In particular, since pairing devices may require a user to perform multiple manual steps in order to complete the pairing process, avoiding such a pairing process can help to reduce the overall burden to the user. In addition, since pairing devices typically results in the computing device retaining a record of the previously-paired device to facilitate easier re-pairing in the future, avoiding such a pairing process can reduce usage of memory in the computing device-this may be particularly helpful if, for example, the user is expected to use many separate medication delivery devices over a relatively short period. The inventors have further appreciated that using advertising packets, such as Bluetooth Low Energy (BLE) advertising packets, may help to improve the battery life of a battery-powered medication delivery device, as opposed to other wireless communication options.

As described herein, the inventors have appreciated that it is desirable for a computing device and the medication delivery device to establish a wireless communication session without using a formal connection process (e.g., without establishing a formal Bluetooth paired session using a standard Bluetooth handshake protocol with the user's smartphone). For example, as described above, using a standard Bluetooth pairing protocol causes a smartphone or other computing device to automatically save previously-paired devices in a list of Bluetooth paired devices. Over time, this can result in an undesirably large number of Bluetooth paired devices in that list, which can make it difficult for a user to identify the particular device of interest, can consume a large amount of computing resources of the computing device, and/or the like. As another example, the formal Bluetooth pairing session can take a non-negligible amount of time (e.g., 15-30 seconds) to establish. Since user's may need to pair new medication delivery devices regularly, it is therefore desirable to minimize the pairing time to decrease the burden on the user. The inventors have also determined that it can be desirable to use custom encryption techniques (e.g., different than those provided by the Bluetooth protocol). Accordingly, the inventors developed a custom connection protocol that can be used to connect a computing device with a medication delivery device.

The inventors have developed techniques to connect a computing device and a medication delivery device that addresses at least some of the aforementioned issues. In some embodiments, the techniques include (a) receiving a device identifier (e.g., a serial number) associated with the medication delivery device at a computing device associated with a user, (b) determining, based on the device identifier, whether the medication delivery device is associated with a different user other than the user (e.g., by checking with a database of existing entries), (c) upon determining that the medication delivery device is not associated with the different user, storing information associated with the medication delivery device at the computing device, which includes at least an encryption key, (d) receiving, at the computing device, encrypted information (e.g., status information, dosage information) associated with the medication delivery device, and (e) using the encryption key to decrypt the encrypted information associated with the medication delivery device.

The inventors have recognized that it is desirable to instruct users that, prior to using the medication delivery device, the user should ideally first register or associate the medication delivery device to the user's account (e.g., to monitor dose information, expiration information, etc.). However, the inventors have also appreciated that it is desirable to not actually prevent use of the medication delivery device prior to it being associated to the user's account. As a result, the inventors have appreciated a need to detect when a medication delivery device is used prior to being associated with a user's account, and to notify the user that the device that is being used or was used is not registered. It can also be desirable to instruct the user to associate the medication delivery device with the user account. The inventors have developed techniques to determine if a medication delivery device is associated with a user account, and to send a warning message if the medication delivery device is determined to be used even if the device is not associated with a user account without preventing the user from using the medication delivery device. In some embodiment, the techniques include (i) receiving, from the medication delivery device, a message comprising: (a) identification information for the medication delivery device; and (b) dose information indicative of a number of doses administered by the medication delivery device; (ii) determining, based on the identification information of the medication delivery device, whether the medication delivery device is associated with any user account; and (iii) upon determining that the medication delivery device is not associated with any user account, displaying an error message indicating that the medication delivery device should be associated with an account of a user of the medication delivery device, without preventing the user from using the medication delivery device prior to associating the medication delivery device with the account.

The inventors have also recognized that there may be challenges associated with processing information from a particular medication delivery device. For example, when there are multiple medication delivery devices within range of the computing device (e.g., within range of the user's smartphone), it may be challenging to distinguish between information originating at one device, as compared to another. For example, such a situation may arise when the user owns multiple medication delivery devices or if the user is in a facility, such as a public restroom, where there are other users of medication delivery devices. As a result, when a user is attempting to view information relating to a particular medication delivery, the computing device may incorrectly present information from another device or not present information at all.

While the user could physically separate the computing device and the additional medication delivery devices such that they are out-of-range of one another, this would place an unnecessary burden on the user. For example, if the user lives in a small home, establishing a sufficient physical separation may require the user to leave their home. Additionally or alternatively, if the user is attempting to query a medication delivery device in a public setting, in a hospital, in a nursing home, etc., the user may not be able to control the separation between their computing device and medication delivery devices belonging to other users.

Accordingly, the inventors have developed techniques that address the above-described challenges associated with presenting information from a particular medication delivery device. The techniques can include determining if packets are being received from multiple medication delivery devices, and if so, obtaining information on the medication delivery device of interest to determine status information of just the device of interest. For example, the techniques can include prompting, via a user interface, a user to input identifying information of the medication delivery device of interest. The user can, for example, input at least part of a serial number or other identifying information of the medication delivery device of interest through a user interface. As another example, the user can scan a barcode (e.g., a QR code) associated with the medication delivery device.

In some embodiments, the techniques include (a) receiving a set of advertising packets, including at least one advertising packet from one or more medication delivery devices, (b) determining a number of unique identifiers included in the set of advertising packets to determine a number of transmitting devices, (c) determining, based on the number of transmitting medication delivery devices, whether to prompt the user to provide identification information for the particular medication delivery device; and (d) presenting the information from the particular medication delivery device to the user.

As described herein, connecting medication delivery devices to a computing device (e.g., a smartphone) can, over time, result in an undesirably large number of devices in an associated device list (e.g., a list of devices paired over Bluetooth). Such a long list can be cumbersome to navigate by a user and/or consume unnecessary computing resources (e.g., when the listed devices are no longer in use). The inventors have recognized that it is desirable to retire a medication delivery device under certain circumstances (e.g., when the device has been in use for more than a predetermined number of days, is empty, has a low battery level, and/or the like). The inventors have therefore developed techniques for retiring a medication delivery device, such as by deleting the medication delivery device from a list of devices and/or moving the medication delivery device's entry from the list to a separate list (e.g., a hidden list and/or a list of inactive medication delivery devices). In some embodiments of a medication delivery device comprising a reservoir sized sufficiently to hold medication for a plurality of doses and a wireless communication unit that wirelessly transmits information about the medication delivery device, the techniques include (i) determining, based on the accessed data, to retire the medication delivery device; and (ii) retiring the medication delivery device; wherein retiring the medication delivery device comprises at least one of: removing the medication delivery device from a list of active medication delivery devices associated with the user account and (b) moving an entry for the medication delivery device from the list of active medication delivery devices associated with the user account to a list of retired medication delivery devices.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

Devices described herein may comprise a medication, such as for example, within a reservoir or cartridge 20 (described below). In another embodiment, a system may comprise one or more devices including device 10 (described below) and a medication. The term “medication” refers to one or more therapeutic agents including but not limited to insulins, insulin analogs such as insulin lispro or insulin glargine, insulin derivatives, GLP-1 receptor agonists such as dulaglutide or liraglutide, glucagon, glucagon analogs, glucagon derivatives, gastric inhibitory polypeptide (GIP), GIP analogs, GIP derivatives, oxyntomodulin analogs, oxyntomodulin derivatives, therapeutic antibodies and any therapeutic agent that is capable of delivery by the devices described herein. The medication as used in the device may be formulated with one or more excipients. The device is operated in a manner generally as described above by a patient, caregiver or healthcare professional to deliver medication to a person.

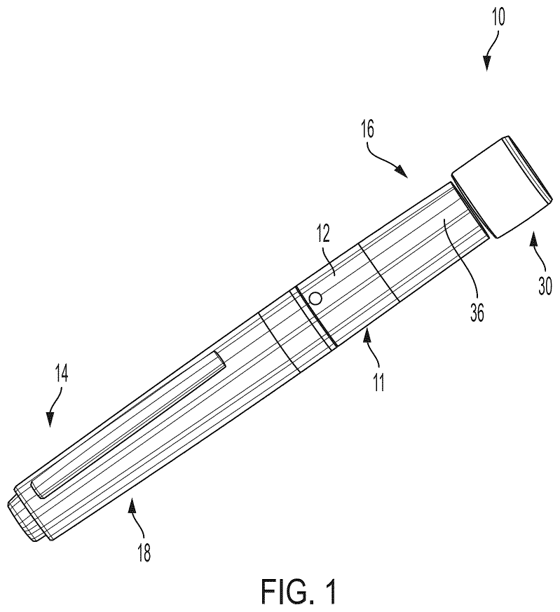

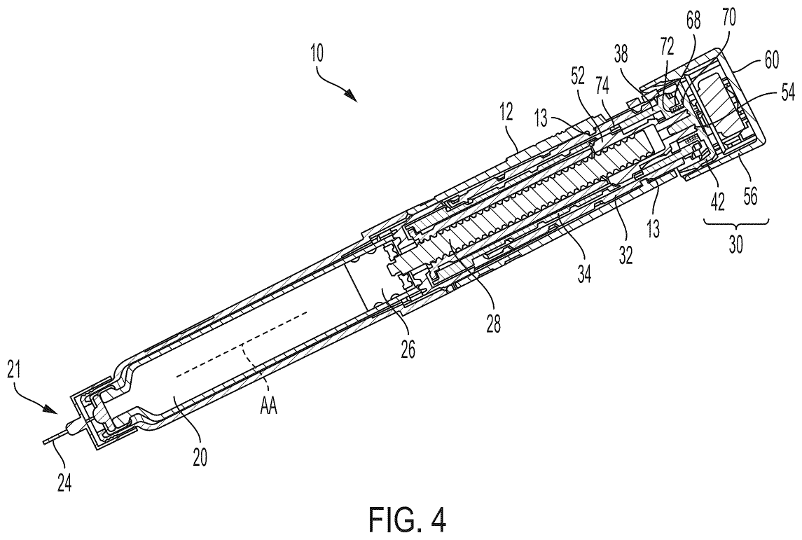

An exemplary medication delivery device 10 is illustrated in FIGS. 1-4 as a pen injector configured to inject a medication into a patient through a needle. Device 10 includes a body 11 that may comprise an elongated, pen-shaped housing 12 including a distal portion 14 and a proximal portion 16. As used herein, the term “distal” refers to the direction and/or portion of a medication delivery device that is pointed towards (or located closer to) the site of injection, while the term “proximal” refers to the direction and/or portion of a medication delivery device that is pointed away from (or located further away from) the site of injection. Distal portion 14 may be received within a pen cap 18. Referring to FIG. 4, distal portion 14 may contain a reservoir or cartridge 20 configured to hold medication to be dispensed through the outlet 21 of the housing a dispensing operation. The outlet 21 of distal portion 14 may be equipped with an injection needle 24. In some embodiments, the injection needle is removable from the housing. In some embodiments, the injection needle is replaced with a new injection needle after each use.

A piston 26 may be positioned in reservoir 20. The medication delivery device may include an injecting mechanism positioned in proximal portion 16 that is operative to advance piston 26 toward the outlet of reservoir 20 during the dose dispensing operation to force the contained medicine through the needled end. The injecting mechanism may include a drive member 28, illustratively in the form of a screw, that is axially moveable relative to housing 12 to advance piston 26 through reservoir 20.

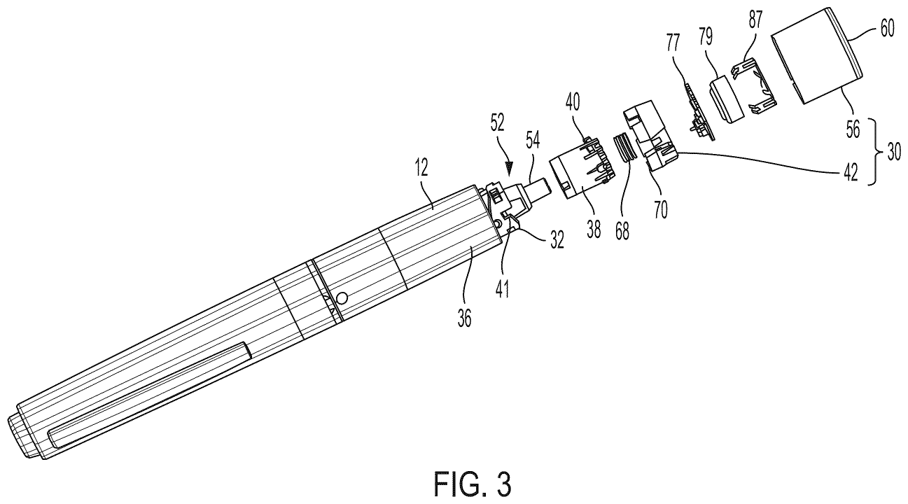

The device may include a dose-setting assembly coupled to the housing 12 for setting a dose amount to be dispensed by device 10. As best seen in FIGS. 3 and 4, in the illustrated embodiment, the dose-setting assembly includes a dose-setting screw 32 and a flange 38. The dose-setting screw 32 is in the form of a screw element operative to spiral (i.e., simultaneously move axially and rotationally) about a longitudinal axis AA of rotation relative to housing 12 during dose setting and dose dispensing. FIGS. 3 and 4 illustrate the dose-setting screw 32 fully screwed into housing 12 at its home or zero dose position. Dose-setting screw 32 is operative to screw out in a proximal direction from housing 12 until it reaches a fully extended position corresponding to a maximum dose deliverable by device 10 in a single injection. The extended position may be any position between a position corresponding to an incremental extended position (such as a dose setting a 0.5 or 1 unit) to a fully extended position corresponding to a maximum dose deliverable by device 10 in a single injection and to screw into housing 12 in a distal direction until it reaches the home or zero position corresponding to a minimum dose deliverable by device 10 in a single injection.

Referring to FIGS. 3 and 4, dose-setting screw 32 includes a helically threaded outer surface that engages a corresponding threaded inner surface 13 of housing 12 to allow dose-setting screw 32 to spiral (i.e. simultaneously rotate and translate) relative to housing 12. Dose-setting screw 32 further includes a helically threaded inner surface that engages a threaded outer surface of sleeve 34 (FIG. 4) of device 10. The outer surface of dose-setting screw 32 includes dose indicator markings, such as numbers that are visible through a dosage window 36 to indicate to the user the set dose amount.

As mentioned above, in some embodiments, the dose-setting assembly further includes a tubular flange 38 that is coupled in the open proximal end of dose-setting screw 32 and is axially and rotationally locked to the dose-setting screw 32 by protrusions 40 received within openings 41 in the dose-setting screw 32. The protrusions 40 of the flange 38 can be seen in FIGS. 3, 8 and 9, and the openings 41 of the dose-setting screw 32 can be seen in FIG. 3.

As seen in FIGS. 3 and 4, delivery device 10 may include an actuator assembly having a clutch 52 and a dose button 30. The clutch 52 is received within the dose-setting screw 32, and the clutch 52 includes an axially extending stem 54 at its proximal end. The dose button 30 of the actuator assembly is positioned proximally of the dose-setting screw 32 and flange 38. Dose button 30 includes a support 42, also referred to herein as an “under button,” and a cover 56, also referred to herein as an “over button.” As will be discussed, the support 42 and cover 56 enclose electronics components used to store and/or communicate data relating to amount of dose delivered by a medication delivery device.

The support 42 of the dose button may be attached to the stem 54 of the clutch 52, such as with an interference fit or an ultrasonic weld, so as to axially and rotatably fix together dose button 30 and clutch 52.

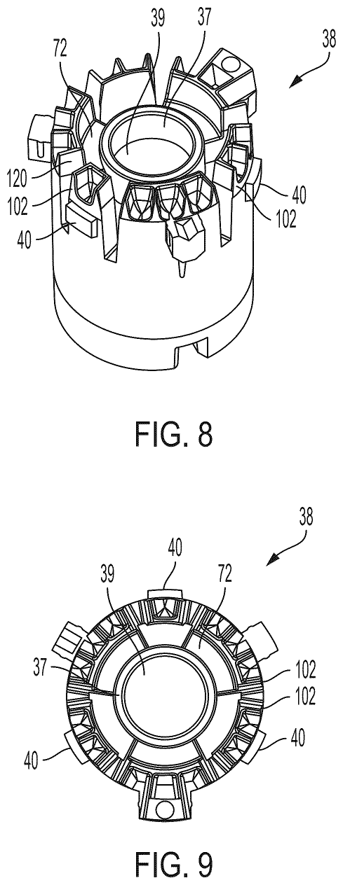

In some embodiments, a portion of the clutch may pass through a lumen 39 of the flange 38. The lumen 39 of the flange is best seen in FIGS. 8 and 9. The lumen 39 may, in some embodiments, serve to help center the clutch 52 in place.

Proximal face 60 of the dose button 30 may serve as a push surface against which a force can be applied manually, i.e., directly by the user to push the actuator assembly (dose button 30 and clutch 52) in a distal direction. A bias member 68, illustratively a spring, may be disposed between the distal surface 70 of support 42 and a proximal surface 72 of tubular flange 38 (FIGS. 8 and 9) to urge the support 42 of the actuation assembly and the flange 38 of the dose-setting assembly axially away from each other. Dose button 30 is depressible by a user to initiate the dose dispensing operation. In some embodiments, the bias member 68 is seated against this proximal surface 72 and may surround a raised collar 37 of the flange 38.

Delivery device 10 is operable in a dose setting mode and a dose dispensing mode. In the dose setting mode of operation, the dose button 30 is rotated relative to housing 12 to set a desired dose to be delivered by device 10. In some embodiments, rotating the dose button 30 in one direction relative to the housing 12 causes the dose button 30 to axially translate proximally relative to the housing 12, and rotating the dose button 30 in the opposite direction relative to the housing 12 causes the dose button 30 to axially translate distally relative to the housing. In some embodiments, clockwise rotation of the dose button moves the dose button 30 distally, and counter-clockwise rotation of the dose button moves the dose button proximally, or vice versa.

In some embodiments, rotating the dose button 30 to axially translate the dose button 30 in the proximal direction serves to increase the set dose, and rotating the dose button 30 to axially translate the dose button 30 in the distal direction serves to decrease the set dose. The dose button 30 is adjustable in pre-defined rotational increments corresponding to the minimum incremental increase or decrease of the set dose during the dose setting operation. The dose button may include a detent mechanism such that each rotational increment produces an audible and/or tactile “click.” For example, one increment or “click” may equal one-half or one unit of medication.

In some embodiments, the set dose amount may be visible to the user via the dial indicator markings shown through a dosage window 36. During the dose setting mode, the actuator assembly, which includes the dose button 30 and clutch 52, moves axially and rotationally with the dose-setting assembly, which includes the flange 38 and the dose-setting screw 32.

Dose-setting screw 32 and flange 38 are fixed rotationally to one another, and rotate and move proximally during dose setting, due to the threaded connection of the dose-setting screw 32 with housing 12. During this dose setting motion, the dose button 30 is rotationally fixed relative to the flange 38 and the dose-setting screw 32 by complementary splines 74 of flange 38 and clutch 52 (FIG. 4), which are urged together by the bias member 68. In the course of dose setting, the dose-setting screw 32, flange 38, clutch 52, and dose button 30 move relative to the housing 12 in a spiral manner (i.e. simultaneous rotation and axial translation) from a “start” position to an “end” position. This rotation and translation relative to the housing is in proportion to the amount of dose set by operation of the medication delivery device 10.

Once the desired dose is set, device 10 is manipulated so the injection needle 24 properly penetrates, for example, a user's skin. The dose dispensing mode of operation is initiated in response to an axial distal force applied to the proximal face 60 of dose button 30. The axial force is applied by the user directly to dose button 30. This causes axial movement of the actuator assembly (dose button 30 and clutch 52) in the distal direction relative to housing 12.

The axial shifting motion of the actuator assembly compresses biasing member 68 and reduces or closes the gap between dose button 30 and the tubular flange 38. This relative axial movement separates the complementary splines 74 on clutch 52 and flange 38, and thereby disengages the dose button 30 from being rotationally fixed to the flange 38 and the dose-setting screw 32. In particular, the dose-setting screw 32 is rotationally uncoupled from the dose button 30 to allow backdriving rotation of the dose-setting screw 32 relative to the dose button 30 and the housing 12. Also, while the dose-setting screw 32 and flange 38 are free to rotate relative to the housing 12, the dose button 30 is held from rotating relative to the housing 12 by the user's engagement of dose button 30 by pressing against it.

As dose button 30 and clutch 52 are continued to be axially plunged without rotation relative to housing 12, dose-setting screw 32 screws back into housing 12 as it spins relative to dose button 30. The dose markings that indicate the amount still remaining to be injected are visible through window 36. As dose-setting screw 32 screws down distally, drive member 28 is advanced distally to push piston 26 through reservoir 20 and expel medication through needle 24.

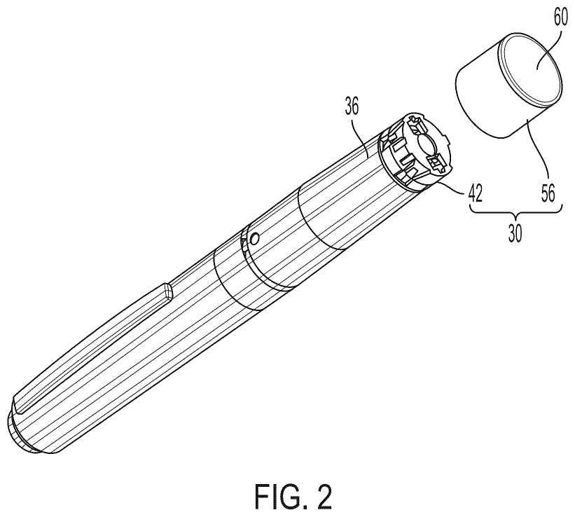

During the dose dispensing operation, the amount of medicine expelled from the medication delivery device is proportional to the amount of rotational movement of the dose-setting screw 32 relative to the housing 12 as the dose-setting screw 32 screws back into housing 12. In some embodiments, because the dose button 30 is rotationally fixed relative to the housing 12 during the dose dispensing mode, the amount of medicine expelled from the medication delivery device may be viewed as being proportional to the amount of rotational movement of the dose-setting screw 32 relative to the dose button 30 as the dose-setting 32 screws back into housing 12. The injection is completed when the internal threading of dose-setting screw 32 has reached the distal end of the corresponding outer threading of sleeve 34 (FIG. 4). Device 10 is then once again arranged in a ready state or zero dose position as shown in FIGS. 2 and 4.

As discussed above, the dose delivered may be derived based on the amount of rotation of the dose-setting assembly (flange 38 and dose-setting screw 32) relative to the actuator assembly (clutch 52 and dose button 30) during dose delivery. This rotation may be determined by detecting the incremental movements of the dose-setting assembly which are “counted” as the dose-setting assembly is rotated during dose delivery.

Further details of the design and operation of an exemplary delivery device 10 may be found in U.S. Pat. No. 7,291,132, entitled Medication Dispensing Apparatus with Triple Screw Threads for Mechanical Advantage, the entire disclosure of which is hereby incorporated by reference herein. Another example of the delivery device is an auto-injector device that may be found in U.S. Pat. No. 8,734,394, entitled “Automatic Injection Device With Delay Mechanism Including Dual Functioning Biasing Member,” which is hereby incorporated by reference in its entirety, where such device being modified with one or more various sensor systems described herein to determine an amount of medication delivered from the medication delivery device based on the sensing of relative rotation within the medication delivery device. Another example of the delivery device is a reusable pen device that may be found in U.S. Pat. No. 7,195,616, entitled “Medication Injector Apparatus with Drive Assembly that Facilitates Reset,” which is hereby incorporated by reference in its entirety, where such device being modified with one or more various sensor systems described herein to determine an amount of medication delivered from the medication delivery device based on the sensing of relative rotation within the medication delivery device.

Described herein is a dose detection system that may be operable to determine the amount of dose delivered based on relative rotation between a dose setting member and the device body. The dose detection system utilizes a dose setting member attached to the device body and rotatable relative to the device body about an axis of rotation during dose delivery. A sensed element is attached to and rotationally fixed with the dose setting member. An actuator is attached to the device body and is held against rotation relative to the device body during dose delivery. The sensed element thereby rotates relative to the actuator during dose delivery in relation to the amount of dose delivered.

In some embodiments, the dose detection system comprises a rotational sensor attached to the actuator assembly and a sensed element that includes surface features that are equally radially spaced about the axis of rotation of the sensed element.

In some embodiments, the dose detection systems may include a sensor and a sensed component attached to components of the medication delivery device. The term “attached” encompasses any manner of securing the position of a component to another component or to a member of the medication delivery device such that they are operable as described herein. For example, a sensor may be attached to a component of the medication delivery device by being directly positioned on, received within, integral with, or otherwise connected to, the component. Connections may include, for example, connections formed by frictional engagement, splines, a snap or press fit, sonic welding or adhesive.

The term “directly attached” is used to describe an attachment in which two components, or a component and a member, are physically secured together with no intermediate member, other than attachment components. An attachment component may comprise a fastener, adapter or other part of a fastening system, such as a compressible membrane interposed between the two components to facilitate the attachment. A “direct attachment” is distinguished from attachment where the components/members are coupled by one or more intermediate functional members.

The term “fixed” is used to denote that an indicated movement either can or cannot occur. For example, a first member is “fixed rotationally” with a second member if the two members are required to move together in rotation. In one aspect, a member may be “fixed” relative to another member functionally, rather than structurally. For example, a member may be pressed against another member such that the frictional engagement between the two members fixes them together rotationally, while the two members may not be fixed together absent the pressing of the first member.

Various sensor arrangements are contemplated herein. In general, the sensor arrangements comprise a sensor and a sensed component. The term “sensor” refers to any component which is able to detect the relative position or movement of the sensed component. The sensor may be used with associated electrical components to operate the sensor. The “sensed component” is any component for which the sensor is able to detect the position and/or movement of the sensed component relative to the sensor. For the dose detection system, the sensed component rotates relative to the sensor, which is able to detect the rotational movement of the sensed component. The sensor may comprise one or more sensing elements, and the sensed component may comprise one or more sensed elements. The sensor detects the movement of the sensed component and provides outputs representative of the movement of the sensed component.

Illustratively, the dose detection system includes an electronics assembly suitable for operation of the sensor arrangement as described herein. The medication delivery device may include a controller that is operably connected to the sensor to receive outputs from the sensor. The controller begins receiving generated signals from the sensor indicative of counts from first to last one for a total number of counts that is used for determining total displacement, e.g. angular displacement. In the case of detecting an angular movement of a dose-setting assembly, the controller may be configured to receive data indicative of the angular movement of the dose-setting assembly that can be used to determine from the outputs the amount of dose delivered by operation of the medication delivery device. The controller may be configured to determine from the outputs the amount of dose delivered by operation of the medication delivery device. Alternatively, the controller may communicate the data indicative of the angular movement to another device in order to allow the other device to determine the amount of dose delivered. The controller may include conventional components such as a processor, power supply, memory, microcontrollers, etc. Alternatively, at least some components may be provided separately, such as by means of a computer, smart phone or other device. Means are then provided to operably connect the external controller components with the sensor at appropriate times, such as by a wired or wireless connection.

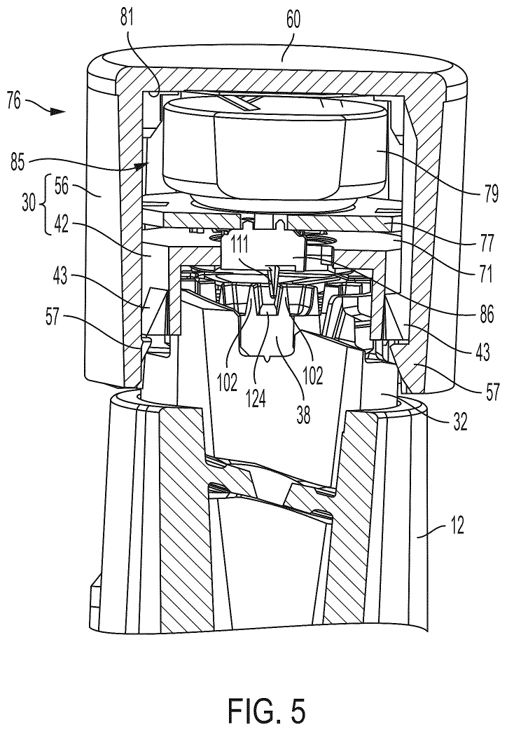

According to one aspect, the electronics assembly includes a sensor arrangement including one or more sensors operatively communicating with a processor for receiving signals from the sensor representative of the sensed rotation. An exemplary electronics assembly 76 is shown in FIGS. 5-7 and can include a sensor 86, and a printed circuit board (PCB) 77 having a plurality of electronic components. The printed circuit board may be a flexible printed circuit board. The circuit board of the electronics assembly 76 may include a microcontroller unit (MCU) as the controller comprising at least one processing core and internal memory. The electronics assembly may include a power source 79, e.g. a battery, illustratively a coin cell battery, for powering the components. The controller of electronics assembly 76 may include control logic operative to perform the operations described herein, including detecting the angular movement of the dose-setting assembly during dose setting and/or dose delivery and/or detecting a dose delivered by medication delivery device 10 based on a detected rotation of the dose-setting assembly relative to the actuator assembly. Many, if not all of the components of the electronics assembly, may be contained in a compartment 85 within the dose button 30. In some embodiments, the compartment 85 may be defined between a proximal surface 71 of support 42 of the dose button and a distal surface 81 of the cover 56 of the dose button. In the embodiment shown in FIG. 5, the electronics assembly 76 is permanently integrated within the dose button 30 of the delivery device. In other embodiments, the electronics assembly is provided as a module that can be removably attached to the actuator assembly of the medication delivery device.

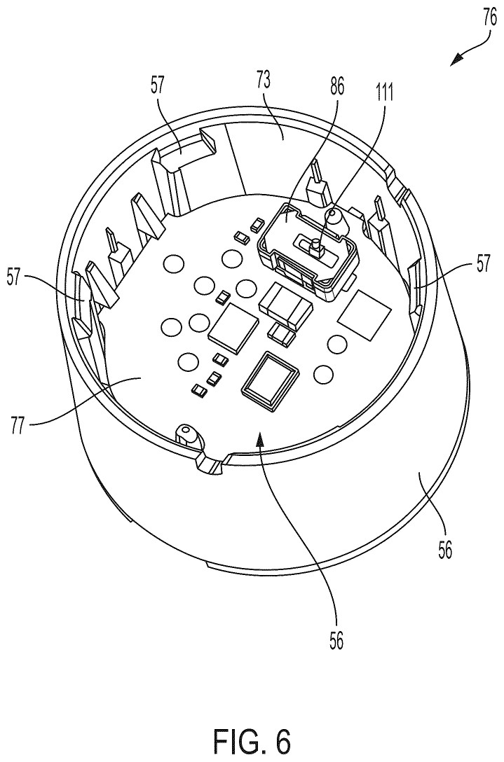

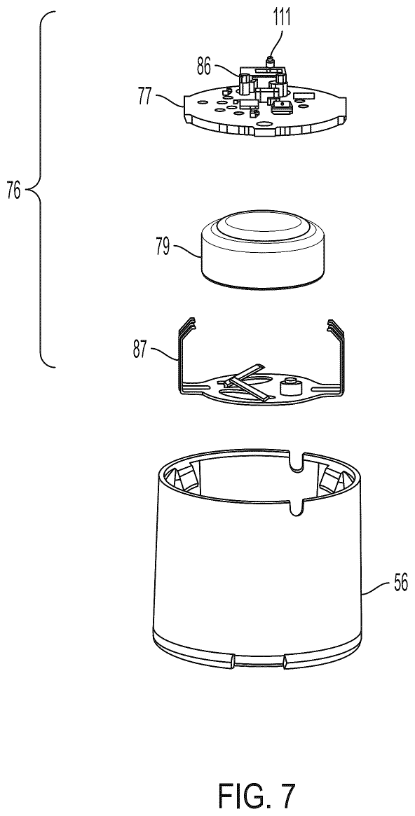

An underside view of the electronics assembly 76 held within the cover 56 is shown in FIG. 6, and an exploded view of the electronics assembly 76 is shown in FIG. 7. As shown in FIGS. 6 and 7, the electronics assembly 76 may include a printed circuit board (PCB) 77 and a sensor 86 having a contact surface 111. As shown in FIG. 7, the electronics assembly 76 may also include a battery 79 and a battery cage 87.

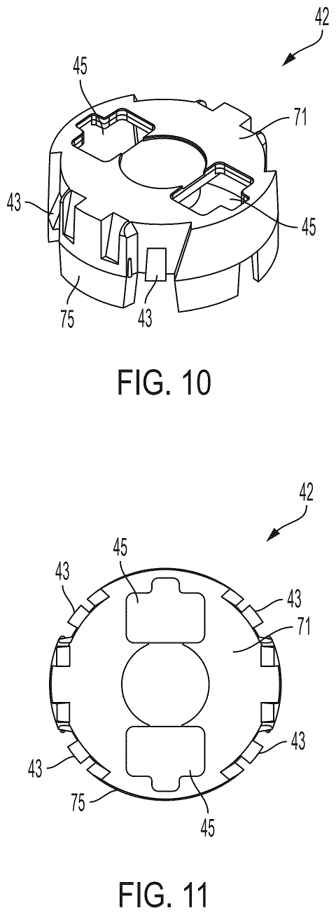

In some embodiments, at least a portion of the sensor 86 extends out of the compartment 85 of the dose button 30. As best seen in FIGS. 10 and 11, the support 42 of the dose button 30 may include one or more openings 45 through which the sensor 86 can extend through. In some embodiments, during assembly of the medication delivery device, the contact surface 111 of the sensor 86 is passed through the opening 45 of the support 42. This may permit the contact surface 111 of the sensor to interact with a component that is external to the compartment 85 of the dose button 30. In some embodiments, while only one of the openings 45 in the support 42 is needed to accommodate a sensor, a second opening may be provided, e.g. for symmetry of the support component, which help with manufacturing of the component and/or assembly of the component with the medication delivery device.

The controller of electronics assembly 76 may be operative to store the total angular movement used for determining dose delivery and/or the detected dose delivery in local memory (e.g., internal flash memory or on-board EEPROM). The controller may be further operative to wirelessly transmit a signal representative of the total counts, total angular movement, and/or detected dose to an external device, such as a user's mobile device or a remote server. Transmission may, for example, be over a Bluetooth low energy (BLE) or other suitable short or long range wireless communication protocol. Illustratively, the BLE control logic and controller are integrated on the same circuit.

As discussed, according to one aspect, the dose detection system involves detecting relative rotational movement between two assemblies of the medication delivery device. With the extent of rotation having a known relationship to the amount of a delivered dose, the sensor operates to detect the amount of angular movement from the start of a dose injection to the end of the dose injection. For example, in some embodiments, the relationship for a pen injector is that an angular displacement of a dose-setting assembly of 18° is the equivalent of one unit of dose, although other angular relationships are also suitable, such as, for example, 9, 10, 15, 20, 24 or 36 degrees may be used for a unit or a half unit. The sensor system is operable to determine the total angular displacement of a dose setting member during dose delivery. Thus, if the angular displacement is 90°, then 5 units of dose have been delivered.

The angular displacement is determined by counting increments of dose amounts as the injection proceeds. For example, a sensing system may use a repeating pattern of a sensed element, such that each repetition is an indication of a predetermined degree of angular rotation. Conveniently, the pattern may be established such that each repetition corresponds to the minimum increment of dose that can be set with the medication delivery device.

The dose detection system components may be permanently or removably attached to the medication delivery device. In some embodiments, at least some of the dose detection system components are provided in the form of a module that is removably attached to the medication delivery device. In other embodiments, the dose detection system components are permanently attached to the medication delivery device.

In some embodiments, a sensor may detect, during dose delivery, the relative rotation of a sensed component that is rotationally fixed to the dose-setting screw 32, from which is determined the amount of a dose delivered by the medication delivery device. In an illustrative embodiment, a rotational sensor is attached, and rotationally fixed, to the actuator assembly. The actuator assembly does not rotate relative to the device housing during dose delivery.

In some embodiments, a sensed component is attached, and rotationally fixed, to the dose-setting screw 32, which rotates relative to the dose button 30 and the device housing 12 during dose delivery. In some of the embodiments described herein, the sensed component includes a ring structure having a plurality of proximally extending projections circumferentially disposed relative to one another. Projections are shaped and sized to deflect a movable element of the rotational sensor. One illustrative embodiment of such a sensed component is tubular flange 38, best seen in FIGS. 3, 5, 8, and 9. Embodiments described herein may be provided for a module that is removably attachable to the dose button of the delivery device or integrated within the dose button of the delivery device.

During dose delivery, dose-setting screw 32 is free to rotate relative to dose button 30. In the illustrative embodiment, the electronics assembly 76 is rotationally fixed with the dose button 30 and does not rotate during dose delivery.

As seen in FIGS. 2, 3 and 5, the dose button 30 comprises a cover 56 coupled to a support 42. An electronics assembly 76 may be at least partially contained within a compartment 85 defined between the cover 56 and the support. In some embodiments, the cover and support have corresponding splines that engage with one another to couple the cover and support together. For example, in some embodiments, the cover 56 may couple to the support 42 via one or more snaps 57 on the cover 56 and corresponding to one or more protrusions 43 on the support. As seen in FIGS. 5 and 6, the snaps 57 on the cover 56 may be directed radially inwardly from an inner circumferential sidewall 73. As seen in FIGS. 5, 10 and 11, the protrusions 43 on the support 42 may be directed radially outwardly from an outer circumferential sidewall 75 of the support 42. The protrusions 43 may form a triangular ramp shape.

The snaps 57 on the cover 56 are configured to snap over and mate with the protrusions 43 on the support to couple the cover to the support. In some embodiments, the protrusion on the support comprises a continuous annular protrusion around the outer circumferential sidewall of the support. The cover 56 may attach to the support 42 via frictional engagement, interference fit or any other suitable fit. In some embodiments, the cover 56 is permanently fixed to the support 42 during assembly, e.g. via ultrasonic welding, adhesive, or other suitable fixation approach.

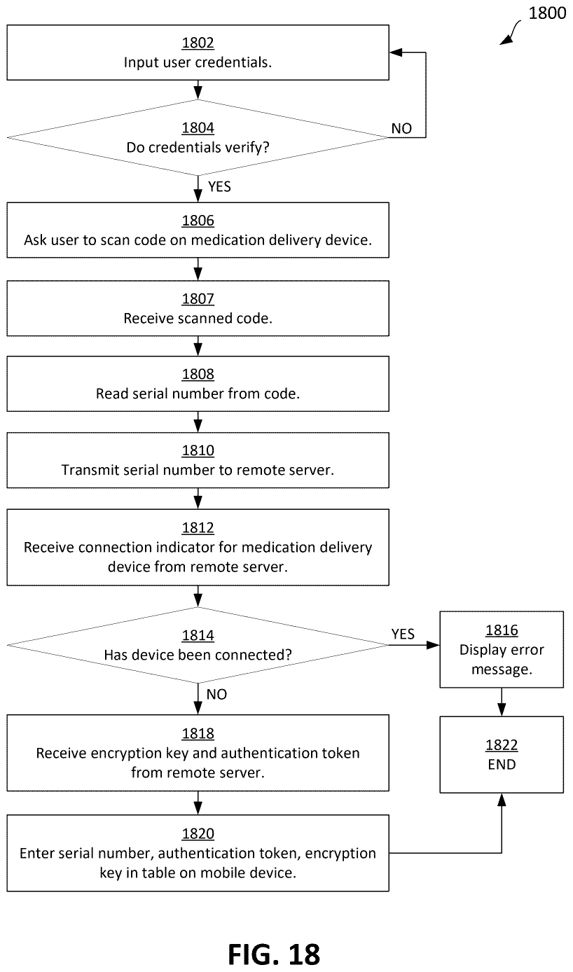

As seen in FIGS. 8 and 9, the tubular flange 38 may include a plurality of axially directed teeth 102 that are equally radially spaced about a rotation axis and arranged to correlate to the equivalent of one unit of dose. In this illustrative embodiment, the tubular flange 38 includes 20 teeth 102 that are equally rotationally spaced from one another, such that the rotation distance between two adjacent teeth corresponds to 18 degrees of rotation. Thus, with the tubular flange 38 of FIGS. 8, 18 degrees of rotation of the tubular flange 38 may be used to represent one dosage unit or a half dosage unit. It should be appreciated that, in other embodiments, different total numbers of teeth may be used to create other angular relationships, such as, for example, 9, 10, 15, 18, 20, 24 or 36 degrees may be used for a unit or 0.5 unit.

A recess 124 may be defined between each pair of adjacent teeth 102. Each tooth 102 may have an approximately triangular shaped profile, each having a surface 120 against which a contact surface 111 of a sensor may slide.

In some embodiments, the sensor for detecting rotation of the tubular flange includes a movable element that has a contact portion capable of resting against the teeth of the tubular flange and is spring-biased such that the contact surface is configured to slide against and over the teeth during rotation of the flange relative to the actuator assembly during dose delivery. The sensor is responsive to the movement of the contact portion over the teeth and generates signals corresponding to the flange. A controller is responsive to the signals generated by the sensor to determine a dose count for determining the dosage delivered based on the detected rotation of the flange relative to the actuator assembly during dose delivery.

The contact surface may be biased against the physical features of the tubular flange to ensure proper contact between the contact surface and the physical features during rotation. In one embodiment, the movable element is a resilient member having one portion attached to the actuator at a location displaced from the contact surface. In one example, the movable element is a following member comprising a beam attached at one end to the actuator and having the contact surface at the other end. The beam is flexed to urge the contact surface in the direction of the surface features. Alternatively, the movable element may be biased in any of a variety of other ways. In addition to the use of a resilient beam, the biasing may be provided, for example, by use of a spring component. Such spring component may for example comprise a compression, tension, or torsion coil spring. In yet other embodiments, the movable element may be biased against the surface features of the sensed element by a separate resilient member or spring component bearing against the movable element.

FIG. 5 depicts an illustrative embodiment of a sensor 86 having a contact surface 111 interacting with teeth 102 of a tubular flange 38. As the flange 38 rotates relative to the dose button 30 during delivery, the teeth 102 of the flange contact and slide against the contact surface 111 of the sensor 86, causing the contact surface 111 to move in an oscillating manner. The movement of the contact surface 111 may be a combination of axial and lateral movement as the contact surface 111 slides into and out of the recesses 124 defined between the teeth 102 of the flange 38. The sensor 86 may be configured to track the movement of the contact surface 111 and associate the movement with an output signal that is sent to a controller.

As alternative to teeth on the tubular flange, surface features that interact with the sensor may comprise anything detectable by the sensor. The sensor arrangement may be based on a variety of sensed characteristics, including tactile, optical, electrical and magnetic properties, for example. In the illustrative embodiments shown in the figures, the surface features are physical features which allow for detection of incremental movements as the dose-setting assembly rotates relative to the actuator assembly. In alternative embodiments, the sensor may be a piezoelectric sensor, a magnetic sensor such as a Hall effect sensor, an accelerometer for detecting vibration, e.g. of a ratcheting or other detent mechanism, where vibration can be correlated with rotational movement, an optical sensor such as a reflective sensor, an interrupter sensor, or an optical encoder, or any other sensor suitable for sensing rotation of a first component relative to a second component.

In some embodiments, when a user presses axially on face 60 of the dose button 30, the dose button 30 advances distally relative to the housing 12, compressing spring 68. Continued pressing of the dose button 30 distally results in back driving of the dose-setting screw 32 in a spiral direction relative to housing 12. As a result, the dose-setting screw 32 and flange 38 are driven to rotate by the axially pressing upon the dose button 30. In some embodiments, the dose detection system is operable for dose detection only while the dose button is being pressed.

In some embodiments, the electronics assembly may include a clock or timer to determine the time elapsed between counts caused by trigger of the rotational sensor from the surface features of the sensed element. When no counts have been detected by the controller after a period of time this may be used to indicate that the dose has completed.

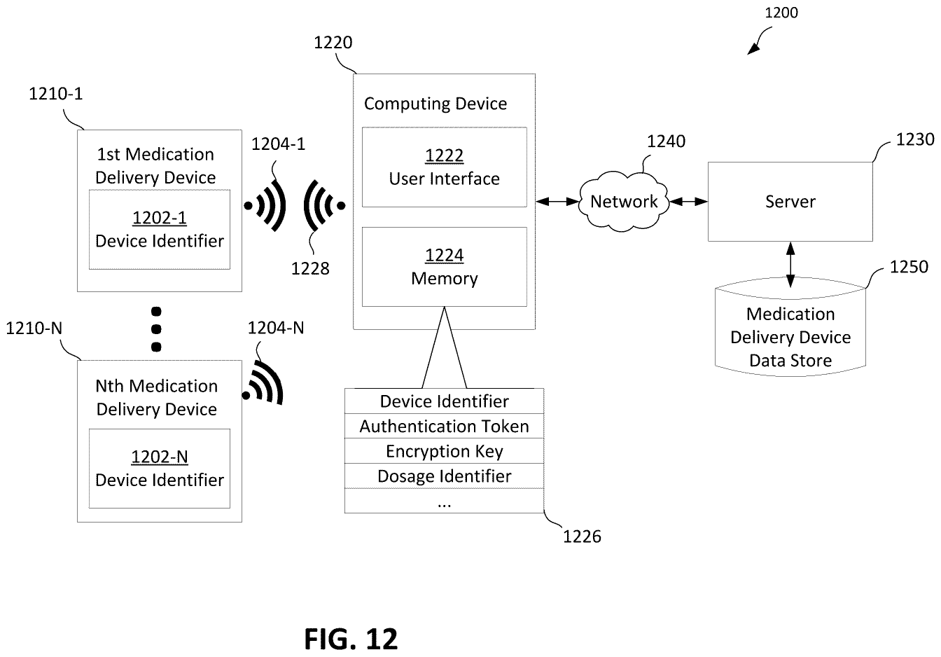

FIG. 12 is a diagram of an example system for wirelessly communicating with a medication delivery device, according to some embodiments. As shown, system 1200 includes medication delivery devices 1210-1 through 1210-N and computing device 1220, which communicates with server 1230 via network 1240. It should be appreciated that system 1200 is illustrative and that a system may have one or more components of any suitable type in addition to or instead of the components illustrated in FIG. 12.

Medication delivery devices 1210-1 through 1210-N may include any suitable number of medication delivery devices. For example, as shown in FIG. 12, the system 1200 includes a first medication delivery device 1210-1 up to and including an Nth medication delivery device 1210-N, where N is any suitable number, as aspects of the technology are not limited in this respect. In some embodiments, though not shown, the medication delivery devices 1210-1 through 1210-N may include only a single medication delivery device such as, for example, only the first medication delivery device 1210-1.

A medication delivery device 1210-1 through 1210-N may include any suitable medication delivery device, such as, for example, an auto-injector or a pre-filled syringe, as aspects of the technology are not limited in this respect. An exemplary medication delivery device 10 is described herein in more detail including at least with respect to FIGS. 1-11. The device described herein is a reusable pen-shaped medication injection device, generally designated, which is manually handled by a user to selectively set a dose and then to inject that set dose. Injection devices of this type are well known, and the description of device is merely illustrative as the sensing system can be adapted for use in variously configured medication delivery devices, including differently constructed pen-shaped medication injection devices, differently shaped injection devices, and infusion pump devices. The medication may be any of a type that may be delivered by such a medication delivery device. Device is intended to be illustrative and not limiting as the sensing system described further below may be used in other differently configured devices.

In some embodiments, one or more of the medication delivery devices 1210-1 through 1210-N are each associated with a unique device identifier. For example, the first medication delivery device 1210-1 is associated with a first device identifier 1202-1, and the Nth medication delivery device 1210-N is associated with an Nth device identifier 1202-N. A device identifier may include any suitable identifier used to distinguish between the medication delivery devices. As nonlimiting examples, a device identifier may include a unique QR Code, a serial number and/or a sequence of characters.

As described herein including at least with respect to FIGS. 5-7, each of the one or more medication delivery devices 1210-1 through 1210-N may include a controller operative to wirelessly transmit a signal using a Bluetooth low energy (BLE) protocol or another suitable short-or long-range wireless communication protocol. In some embodiments, the medication delivery devices 1210-1-1210-N are configured to wirelessly transmit one or more advertising packets. For example, the first medication delivery device 1210-1 may include a controller operative to wirelessly transmit one or more advertising packets 1204-1, and the Nth medication delivery device 1210-N may include a controller operative to wirelessly transmit one or more advertising packets 1204-N. In some embodiments, a medication delivery device transmits such advertising packets continuously and regularly starting at an initial time point, such as the time point at which the medication delivery device is activated, for example.

While some examples provided herein refer to processing BLE advertising packets, it should be appreciated that the techniques can be used to process any wireless packet without departing from the spirit of the techniques described herein. For example, other types of packets that can be processed according to the techniques described herein include packets from any suitable wireless or cellular communication protocols, such as WiFi, Long Range (LoRa), NFC, Radio-Frequency Identification (RFID), Ultra-Wideband (UWB), Matter, Long Term Evolution CAT M1 (LTE-M), Narrowband-Internet of Things (NB-IoT), and 4G. Therefore, such examples are provided for illustrative purposes only and are not intended to limit the techniques described herein.

In some embodiments, advertising packets transmitted by a particular medication delivery device include information associated with the particular medication delivery device. Nonlimiting examples of the types of information that may be included in an advertising packet include a device identifier, a dosage identifier, information indicative of a time associated with a dose of medication delivered using the particular medication delivery device, information indicative of a dosage amount associated with the dose of medication, information indicative of a power level of the particular medication delivery device, information indicative of an amount and/or type of medication held in the particular medication delivery device, information indicative of a time the particular medication delivery device transitioned to a connected state, information indicative of a time the particular medication delivery device was activated, and/or any other suitable type of information as aspects of the technology are not limited in this respect.

In some embodiments, the computing device 1220 is configured to receive a set of advertising packets. For example, the set of advertising packets may include one or more of the advertising packets 1204-1 through 1204-N transmitted by medication delivery devices 1210-1 through 1210-N.

Computing device 1220 may comprise any device that receives, stores, and/or processes information from the medication delivery devices 1210-1 through 1210-N. For example, such information may be received by a communication circuit of the computing device 1220. Exemplary computing devices include a smartphone, a smartwatch, a tablet, and/or a laptop.

In some embodiments, the computing device 1220 includes a user interface 1222 for displaying data and/or receiving user input. For example, the user interface 1222 may comprise a graphical user interface (GUI) including a touchscreen display. The touchscreen display allows the user to interact with presented information, menus, buttons, and other data to provide information to the user or to receive user input from the user. Additionally or alternatively, a keyboard, keypad, microphone, mouse pointer, or other suitable user input device may be provided.

Computing device 1220 may further include a processing circuit or processor(s) 1223 and memory 1224. The processing circuit may take the form of a processor (e.g., a microprocessor or microcontroller, field-programmable gate arrays (FPGAs) and/or digital signal processors (DSPs, or any combination of the foregoing) configured to execute logic stored in a memory to perform the operations described herein. The term “logic”, “control logic”, “instructions” or “application” as used herein may include software and/or firmware executing on any of the aforementioned processing circuits. The memory 1224 may be any suitable computer readable medium that is accessible by the processing circuit and includes both volatile and non-volatile memory. Exemplary memory includes random-access memory (RAM), read-only memory (ROM), electrically erasable programmable ROM (EEPROM), flash memory, a magnetic storage device, optical disk storage, or any other suitable medium which is configured to store data and which is accessible by the processor circuit, whether directly or indirectly via one or more intermediary devices or wired or wireless communication links. Although the preceding description assumes that the memory 1224 is separate from but communicably coupled to the processing circuit, in some embodiments the memory 1224 may also be integrated with the processing circuit. In some embodiments, instead of a processor that executes logic stored in memory, the processing circuit may take the form of hard-wired logic, e.g., a state machine and/or an application-specific integrated circuit (ASIC) that performs the functions described herein.

In some embodiments, memory 1224 is configured to store data table 1226. As nonlimiting examples, data table 1226 may include device identifier(s), authentication token(s), encryption key(s), and/or dosage identifier(s), each of which is described herein in more detail. For example, data table 1226 may comprises a separate entry (e.g., row) for individual medication delivery devices. Each entry may provide the device identifier associated with said individual delivery device (e.g., 1202-1 . . . 1202-N), as well as an authentication token (e.g., 1202-1a, . . . 1202-1Na), an encryption key (1202-1b, . . . 12021Nb), a dosage identifier (1202-1c, . . . 1202-Nc), and/or other information associated with said individual delivery device. The contents and function of said authentication tokens, encryption keys, and/or dosage identifiers are described in further detail herein. In some embodiments, when a medication delivery device has been connected or associated with the user's user account (as described in further detail herein), information pertaining to such a medication delivery device is saved into data table 1226. It should be appreciated, however, that memory 1224 may additionally or alternatively store any other suitable types of information, as aspects of the technology are not limited in this respect.

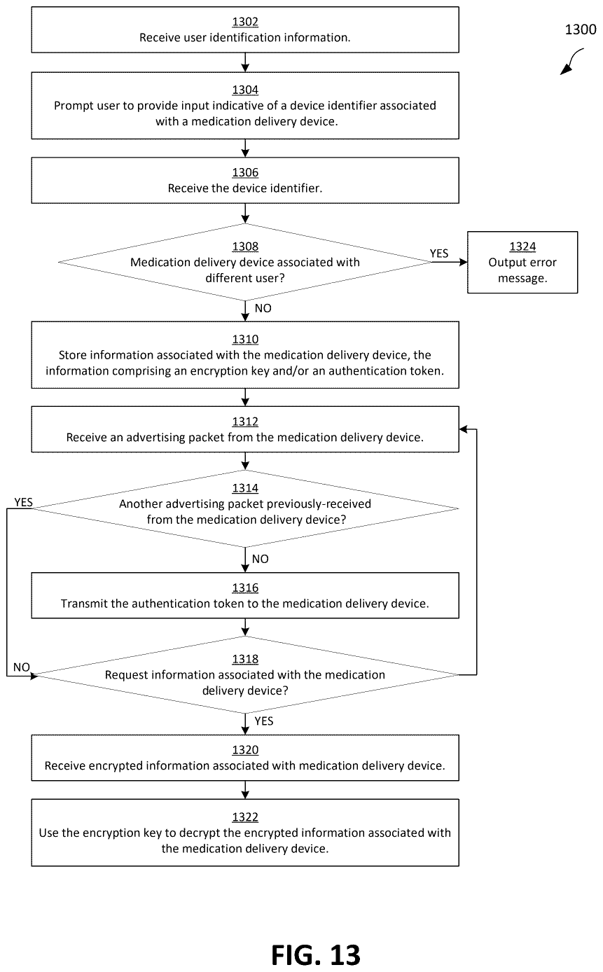

In some embodiments, the computing device 1220 is configured to connect to a medication delivery device according to the connection techniques described herein, including at least with respect to FIG. 13. In some embodiments, connecting to a medication delivery device includes keeping track of device identifiers associated with respective medication delivery devices and/or users. For example, the computing device 1220 may receive information indicative of a device identifier (e.g., through a user interface, in response to scanning a barcode, in response to tapping an NFC tag, etc.) and transmit the device identifier and/or user information associated with the device identifier to server 1230 via network 1240. In some embodiments, server 1230 updates medication delivery device data store 1250 (“data store 1250”) to include the device identifier and/or user information. Accordingly, when the computing device 1220 receives an advertising packet (e.g., advertising packets 1204-1-1204-N) from a medication delivery device with which it has been connected, it can interact with the server 1230 to identify the particular medication delivery device and/or the user with which it is associated.

In some embodiments, server 1230 includes one or multiple computing devices. When server 1230 includes multiple computing devices, the device(s) may be physically co-located (e.g., in a single room) or distributed across multiple physical locations. In some embodiments, server 1230 may be part of a cloud computing infrastructure. In some embodiments, one or more servers 1230 may be co-located in a facility operated by an entity.

In some embodiments, server 1230 maintains and/or is configured to access the data store 1250. In some embodiments, data store 1250 includes any suitable data store, such as a flat file, a data store, a multi-file, or data storage of any suitable type, as aspects of the technology described herein are not limited to any particular type of data store. In some embodiments, data store 1250 stores device identifiers, user information associated with the device identifiers, and any other suitable type of data, although as aspects of the technology describe herein are not limited in this respect. The user information may include, for example, a name, username, password, and/or any other suitable user information. In some embodiments, user information is associated with a particular device identifier stored in the data store 1250. For example, corresponding entries in a table may be populated with the user information and the particular device identifier with which it is associated. However, it should be appreciated that the user information and the device identifiers, and the relationship between them, may be stored in any suitable manner using any suitable data structure(s), as aspects of the technology are not limited in this respect.

Network 1240 may be or include a wide area network (e.g., the Internet), a local area network (e.g., a corporate Internet), and/or any other suitable type of network. Any of the devices shown in FIG. 12 may connect to the network 1240 using one or more wired links, one or more wireless links, and/or any suitable combination thereof. Accordingly, the network 1240 may be, for example, a hard-wired network (e.g., a local area network within a healthcare facility), a wireless network (e.g., connected over Wi-Fi and/or cellular networks), a cloud-based computing network, or any combination thereof.

In some embodiments, server 1230 is further configured to transmit information to computing device 1220 via network 1240. For example, if the computing device 1220 is connecting to a medication delivery device for the first time, the server 1230 may transmit to the computing device 1220 information associated with the medication delivery device (e.g., stored in relation to the device identifier associated with the medication delivery device) such as, for example, an encryption key and/or an authentication token associated with the medication delivery device.

In some embodiments, computing device 1220 transmits information received from the server 1230 to a particular medication delivery device 1210-1-1210-N. For example, if the computing device 1220 is connecting to the first medication delivery device 1210-1 for the first time, then the computing device 1220 may transmit to the first medication delivery device 1210-1 the authentication token associated with the first medication delivery device 1210-1. Transmission may be, for example, over a Bluetooth low energy (BLE) or other suitable short-or long-range wireless communication protocol.

Additionally or alternatively, in some embodiments, computing device 1220 stores the information received from the server 1230 in memory 1224 and may later access the stored information. For example, the first medication delivery device 1210-1 may transmit encrypted information to the computing device 1220 in response to a query from the first medication delivery device 1210-1. Upon receipt of the encrypted information, the computing device 1220 may access, from memory 1224, the encryption key associated with the first medication delivery device 1210-1 and use to the encryption key to decrypt the encrypted information. Additionally or alternatively, such encrypted information may be included in advertising packets received from a medication delivery device, and the encryption key may be used to decrypt such encrypted information.

As described herein, it may be beneficial for a medication delivery device to communicate information to an external computing device associated with a user of the medication delivery device and/or associated with a medical provider. For example, the medication delivery device may provide information regarding a dosage of medication delivered and/or a status of the medication delivery device. The computing device may process the received information to track dose events, plan future dose events, monitor for potential issues, and many other useful applications.

In some embodiments, a medication delivery device may use a standard wireless communication protocol, such as a Bluetooth protocol, to formally “pair” with a computing device. The term “pair” is used herein to refer to the formation of a semi-permanent bond under the Bluetooth protocol. Once paired, the medication delivery device could then transmit dosage and/or status information to the computing device using the wireless protocol.

However, there can be disadvantages associated with establishing a formal paired session between a medication delivery device and a computing device. First, a user may have several medication delivery devices. For example, the user may acquire and use a new medication delivery device every couple of weeks. Each time a formal, paired session is established between a computing device and a new medication delivery device, the computing device automatically saves the medication delivery device in a list of paired devices. Over time, as the user acquires new medication delivery devices, the list of paired devices will grow to an undesirably large number, clogging the memory of the computing device and placing unnecessary burden on the user to maintain the list. Furthermore, a formal pairing session takes time (e.g., 15-30 seconds) to establish, thus increasing the burden on the user, especially when the user is expected to pair new medication delivery devices regularly.

Accordingly, the inventors have developed techniques for connecting a medication delivery device to a computing device without establishing a formal paired session. In some embodiments, a medication delivery device and a computing device are considered to be “connected” once the medication delivery device has been associated with a particular user account.

FIG. 13 is a flowchart showing an exemplary method 1300 for associating or connecting a medication delivery device with a user account, according to some embodiments. In some embodiments, users may be instructed to associate or connect a medication delivery device to his/her user account before, or while, using the delivery device for the first time. For the purposes of explication, method 1300 is described below as being implemented on a user's mobile device, such as computing device 1220. However, this is solely by way of example, as method 1300 may be implemented on any suitable processor. For example, the step(s) may be performed by a laptop computer, a desktop computer, one or more servers, in a cloud computing environment, and/or in any other suitable way.

At step 1302, the user's mobile device receives user identification information. In some embodiments, a user provides the user identification information through a user interface (e.g., user interface 1222 shown in FIG. 12) associated with the mobile device. For example, the user may manually enter the user identification information through a touch screen of the computing device. Additionally or alternatively, the user may verbally provide the user identification information through a microphone of the computing device. Additionally or alternatively, in some embodiments, at least some of the user identification information is stored in memory on the computing device, and the mobile device receives the identification information by accessing the information stored in the memory. However, it should be appreciated that the user identification information may be received using any other suitable techniques, as aspects of the technology described herein are not limited in this respect.

The user identification information, in some embodiments, includes any suitable information used for identifying a user. As nonlimiting examples, the user identification information may include a name, username, password, serial number, address, birth date, healthcare provider information, social security number, or any other suitable identification information, as aspects of the technology described herein are not limited in this respect. In some embodiments, the user's credentials are checked by transmitting them from the user's computing device to a remote server and comparing them at the remote server with a list of users to determine if there is a match. An indicator of the presence or absence of a match can then be transmitted from the remote server back to the user's computing device. If the user's credentials are verified at step 1302, the user is logged into the user's account on the mobile device. In some embodiments, a user account can be unique to a particular user. As a result, the user can use various devices to log into the user's account while still accessing the same account information. In some embodiments, a user may only be allowed to log into an account on a predetermined number of devices (e.g., just on one device, on two devices, etc.). If the user's credentials cannot be verified at step 1302, various actions can be taken. For example, the use can be prompted again to input the proper credentials. As another example, the user's session can be terminated.

At step 1304, the mobile device prompts the user to provide input indicative of a device identifier associated with the medication delivery device. In some embodiments, the mobile device prompts the user to provide the device identifier using any suitable techniques, as aspects of the technology are not limited in this respect. For example, the mobile device may generate a graphical user interface (GUI) that is displayed to the user. The graphical user interface may include elements (e.g., text, a text box, graphics, buttons, etc.) that prompt the user to provide the device identifier through the GUI and/or through another user interface (e.g., a camera, a microphone) associated with the mobile device. Additionally or alternatively, the mobile device may generate audio output through a speaker of the mobile device that prompts the user to provide such input. The device identifier includes any identifier suitable for distinguishing between medication delivery devices, as aspects of the technology are not limited in this respect. As not limiting examples, the device identifier may include a barcode (e.g., a QR code), a serial number, a string of characters, a symbol, or any combination thereof.

At step 1306, the mobile device receives the device identifier from the medication delivery device. In some embodiments, the mobile device receives the device identifier via user input through a user interface. For example, the user may operate a touch screen to enter a serial number or sequence of characters. Additionally or alternatively, in some embodiments, the mobile device receives the device identifier in response to the scanning of a barcode (e.g., a QR code). As an illustrative example, a message can be displayed on the user's computing device asking the user to scan a code affixed or associated with a medication delivery device. For example, a user may operate the camera of a mobile device to scan the barcode. A code can be received from a scan from the medication delivery device, and a serial number for the medication delivery device can be determined based on the scanned code. Additionally or alternatively, in some embodiments, the mobile device receives the device identifier from the medication delivery device itself. For example, the device identifier may be included in an advertising packet, included in information transmitted by an NFC tag on the medication delivery device, or included in information transmitted using any other suitable wireless protocol. However, it should be appreciated that the device identifier may be received through any other suitable means, as aspects of the technology described herein are not limited in this respect.

At step 1308, the mobile device determines whether the medication delivery device is associated with a user other than the user of the mobile device. This step determines whether the medication delivery that the user is seeking to associate with his/her account has actually already been connected with another user's account. In some embodiments, determining whether the medication delivery device is associated with a different user includes transmitting the device identifier to a server (e.g., server 1230 shown in FIG. 12). In some embodiments, the server maintains a data store (e.g., data store 1250 shown in FIG. 12). The data store may store information such as, for example, a list of device identifiers, as well as an indication of whether each device identifier had previously been connected to a user account and/or a computing device. The data store may also store additional information, such as an encryption key unique to the medication delivery device (which may be used to decrypt encrypted data received from the medication delivery device), an authentication token unique to the medication delivery device (which, if transmitted to the medication delivery device, may cause the medication delivery device to transition from an unconnected to a connected state), user identification information associated with the device identifiers, status information for the medication delivery devices associated with the device identifiers, dosage information for the medication delivery device associated with the device identifiers, or any other suitable information. This information may be maintained in the form of a database or table that contains an entry for each medication delivery device, and which provides the above-mentioned information for said delivery device. In response to the above-mentioned query from the mobile device, the server may be configured to check the data store to determine whether the device identifier is already associated with a computing device and/or user identification information. The remote server can then respond to the mobile device based on the results of its query.

In some embodiments, the mobile device may receive from the server an indication that the medication delivery device is associated with a different user. If, at step 1308, the mobile device determines that the medication delivery device is associated with a different user, method 1300 optionally proceeds to step 1324. At step 1324, the mobile device outputs an error message. For example, the mobile device may generate a GUI to be presented to the user, cause audio to be output through a speaker, cause the illumination of one or more light sources, or output the error message by any other suitable means, as aspects of the technology are not limited in this respect. Alternatively, method 1300 may simply end without proceeding to step 1324.

In some embodiments, the mobile device may receive from the server an indication that the medication delivery device is not associated with a different user. For example, the mobile device may receive a message comprising data indicating that the medication delivery device is not associated with a different user. If, at step 1308, the mobile device determines that the medication delivery device is not associated with a different user, method 1300 proceeds to step 1310.

At step 1310, the mobile device can optionally store information associated with the medication delivery device including, for example, information received from the medication delivery device and/or from the server. This information may be stored in, for example, table 1226 at memory 1224. For example, the mobile device may store the device identifier received at step 1306 into the data table 1226 to indicate that the delivery device associated with this device identifier has been connected with the user's account. Additionally or alternatively, as part of step 1310, the mobile device may receive from server 1230 additional information to store in data table 1226, such as the aforementioned unique authentication key and/or encryption key associated with the delivery device. Although not shown here in step 1310, at this point in the logic flow, the server 1230 may also update its data store 1250 to include the device identifier for the medication delivery device, the user identification information (e.g., received at step 1302), and/or any other suitable information associated with the user and the medication delivery device.

In some embodiments, upon completion of the steps 1302-1310, the medication delivery device may be considered associated or connected to the user's user account. For example, creation and storage of the above-mentioned memory entry for the delivery device in the mobile device's local memory (e.g., table 1226 in memory 1224) may be sufficient to connect the medication delivery device to the user account of the user operating the mobile device. If necessary, the mobile device may send a message to server 1230 to inform server 1230 that the medication delivery device should be associated with the user account of the user operating the mobile device, so as to enable server 1230 to update its records accordingly.

However, in some embodiments, additional information stored by the medication delivery device may be required in order to associate or connect a new medication delivery device with the user's account. As a result, the user may be instructed to perform one or more further actions to provide the requisite information to the mobile device so that the medication delivery device can be associated with the user's account.

For example, at step 1311, the mobile device may prompt the user to take some action in order to cause the medication delivery device to start transmitting advertising packets, wherein the advertising packets contain additional information necessary for the mobile device to associate or connect the medication delivery device with the user's account. Such action may include, for example, operating the medication delivery device to expel an initial dose of medication. The medication delivery device may be configured to refrain from transmitting advertising packets until at least a first amount of medication is delivered by the device in order to prevent unnecessary consumption of battery power while the delivery device is in storage or transit before its first use by a user. When the device delivers its first dose of medication, the medication delivery device may be configured to start transmitting said advertising packets at regular or irregular intervals. The medication delivery device may continue transmitting such advertising packets until the device is expressly instructed by an external device to stop transmitting, or until the medication delivery device exhausts its battery. Said first dose may be either a priming dose (e.g., a small dose of medication expelled and discarded to remove any air in the medication delivery pathway of the medication delivery device). Alternatively, said first dose may be a therapeutic dose that is actually injected into the user's body. Either way, delivery of a first dose of medication may cause the medication delivery device to begin transmitting packets (e.g., advertising packets) with the requisite information needed for the mobile device to associate the device with the user's account.

At step 1312, the mobile device receives an advertising packet from the medication delivery device. The advertising packet may include, in some embodiments, a device identifier associated with the medication delivery device, status information, dosage information, a dosage identifier, or any other suitable information as aspects of the technology are not limited in this respect. In some embodiments, the transmitted advertising packets may contain information that authenticates the medication delivery device to the mobile device as a legitimate medication delivery device. For example, memory in the medication delivery device may store a secret encryption key that is unique to the medication delivery device and that is associated with the device identifier received at step 1306. The advertising packet transmitted by the medication delivery device and received by the mobile device at step 1312 may include encrypted data (e.g., an encrypted serial number of the device) that has been encrypted using (i) said secret and unique encryption key and (ii) an initial encryption vector and/or nonce used to perform the encryption, and/or error check information (e.g., information that allows a recipient to check the integrity of the encrypted data, such as a checksum). As discussed in further detail below, this encrypted data may later be used by the mobile device and/or a remote server to authenticate the medication delivery device as a legitimate delivery device.

At step 1314, the mobile device determines whether the medication delivery device that transmitted the advertising packet is already associated or connected with the user's user account (i.e., the user account currently logged into the mobile device). In some embodiments, the mobile device makes this determination by comparing the device identifier included in the received advertising packet against the contents of data table 1226. Data table 1226 may store device identifiers for medication delivery devices from which the mobile device had previously received advertising packets. Alternatively or in addition, data table 1226 may store device identifiers for medication delivery devices that have been successfully connected or associated with the user account currently logged into the mobile device. If the device identifier included in the advertising packet received at step 1312 can be found in data table 1226, the mobile device may conclude that the mobile device had previously received an advertising packet from this particular delivery device before, and/or that this particular delivery device has already been connected or associated with the user's user account (i.e., the user account currently logged into the mobile device). In that case, control may proceed back to step 1312 where the mobile device resumes listening for further advertising packets. If, however, the device identifier included in the advertising packet received at step 1312 cannot be found in table 1226, then the mobile device may conclude that the mobile device had never before received an advertising packet from this particular medication delivery device, and/or that this particular medication delivery device has not yet been connected or associated with the user's user account. In that case, control may proceed to step 1316.

At step 1316, the mobile device verifies the encrypted data in the advertising packet. This may be done by transmitting the encrypted data received from the delivery device to the remote server. The remote server can verify the encrypted data by decrypting the encrypted data using the secret encryption key (which is know only to the medication delivery device and the server) as well as the provided initial encryption vector and/or nonce, and determining whether the decrypted data matches the delivery device's device identifier. Alternatively, the remote server could encrypt the delivery device's identifier using the secret encryption key and the provided initial encryption vector and/or nonce and determine whether the encrypted identifier matches the encrypted data received from the advertising packet. The remote server can then transmit a response back to the mobile device to inform the mobile device whether the encrypted data in the advertising packet could be verified or not. If the mobile device is verified, the remote server can also optionally transmit a unique authentication token and/or encryption key associated with the delivery device, to the extent this information has not already been transmitted as part of step 1310. In some embodiments, a plurality of remote servers can be used for the connection process and/or for user account maintenance. In some embodiments, each remote server can implement some and/or all of the same functionality (e.g., for redundancy). In some embodiments, different server(s) can be used for different functionalities. For example, one or more servers can be used for encryption-related tasks, while one or more different servers can be used for account information and account management.

At step 1318, the mobile device determines, based on the response from the remote server, whether the encrypted data could be verified. If the encrypted data could not be verified, control flows to step 1324 where an error message is output. If the encrypted data is verified, control proceeds to step 1320.

At step 1320, the mobile device connects or associates the medication delivery device to the user account currently logged into the mobile device. This may be done by updating table 1226 at local memory 1224. To the extent this updating was not already done as part of step 1310 (e.g., because the mobile device was configured to authenticate the medication delivery device before adding it to table 1226), a new entry may now be added to table 1226 as part of step 1320. This new entry may be associated with the device identifier received at step 1306 and/or extracted from the advertising packet at step 1312. In addition to the device identifier, the entry may also include the authentication token and/or encryption key received from the remote server at step 1316. The entry may also include a dosage identifier extracted from the advertising packet received at step 1312.

At step 1320, the mobile device may also optionally send a message to the server 1230 indicating that the delivery device associated with the device identifier received at step 1304 should be associated with the user account associated with the user identification information received at step 1302 (to the extent this step was not already completed as part of step 1310).

Also at step 1320, the mobile device can optionally transmit an authentication token to the medication delivery device. In some embodiments, the authentication token is unique to the medication delivery device bearing the unique device identifier received at step 1306. The authentication token may be of any suitable type, as aspects of the technology are not limited in this respect. The authentication token may include any information suitable for establishing uniqueness and/or for identifying a particular medication delivery device, as aspects of the technology are not limited in this respect. The authentication token may be received by the mobile device from the server 1230 as part of the communication from the server to the mobile device described at step 1310, and/or at step 1316. In some embodiments, after receiving the authentication token, the medication delivery device compares the received authentication token to a token stored in the memory of the medication delivery device. If the tokens match, then the medication delivery device stores an indication that it has transitioned from an unconnected state to a connected state. This indicates that the medication delivery device has been successfully associated or registered with a user account. For example, the medication delivery device may flip a flag in its memory to indicate this transition. In some embodiments, after storing an indication that the medication delivery device has transitioned from the unconnected state to the connected state, advertising packets transmitted by the medication delivery device will include information indicative of this transition. For example, the advertising packet may include a binary indication that the medication delivery device is either in an unconnected state or a connected state.

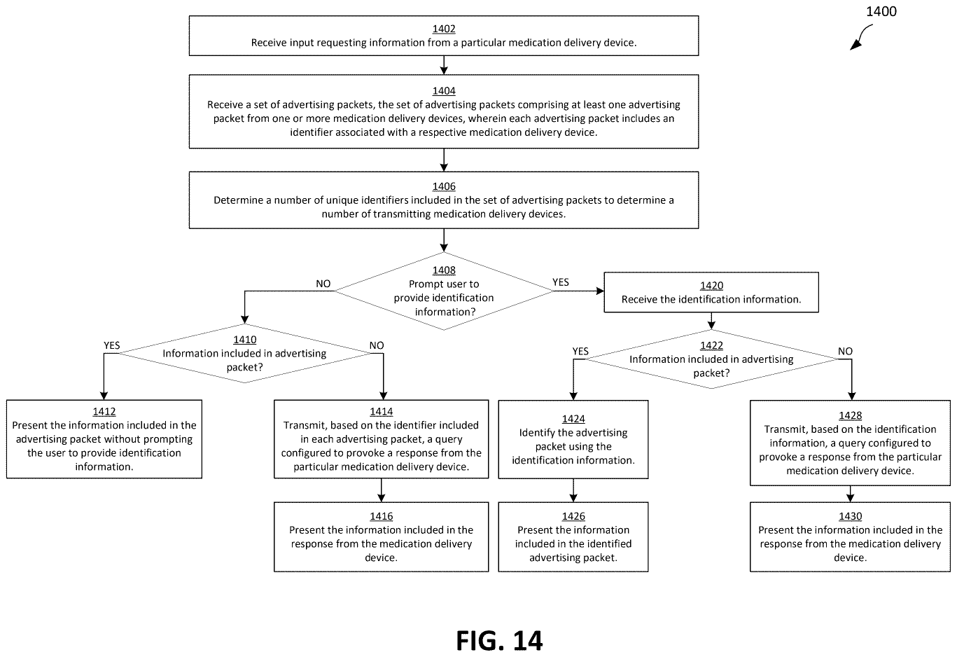

FIG. 14 is a flowchart of an exemplary method 1400 (e.g., executed at a medication delivery device such as 1210-1, 1210-2 . . . 1210-N, etc.) for setting or modifying a flag in a medication delivery device to indicate that the device is associated with a user's account, according to some embodiments. As discussed herein, in some embodiments, authentication data can be used to set or modify a flag in the medication delivery device to store that the device is associated with a user account. For example, the flag may initially be set to indicate a “Not Connected” or “Off” state during manufacturing (e.g., set to indicate a Boolean false value, such as “0”), and may be switched to “Connected” or “On” (e.g., set to indicate a Boolean true value, such as “1”). The flag may be switched, for example,, by transmitting the medication delivery device a (secret) authentication token, which can optionally be transmitted to the medication delivery device as discussed herein. It should be appreciated that various flags and/or data fields can be used to store information on the device that indicates whether the device is associated with a user's account. For example, a flag may initially be set to indicate a “Not Connected” or “Off” state during manufacturing by setting the flag to a Boolean true value (e.g., set to “1”), and the flag may be cleared to indicate a Boolean false value (e.g., set to “0”) to indicate that the device is “Connected” or “On.” As a further example, other types of data fields may be set and/or cleared to indicate the device's connection state in accordance with the techniques described herein.

At step 1402, a message is received by the medication delivery device from the mobile device (e.g., which is optional, as discussed herein). At step 1404, an authentication token is read from the message. At step 1406, the authentication token read from the message is compared with the authentication token stored in the medication delivery device. At step 1408, a determination is made as to whether the authentication token received from the mobile device matches the authentication token stored in the medication delivery device. If it is determined at step 1408 that the two authentication tokens do not match, control proceeds to step 1410, where the process ends. If it is determined at step 1408 that the two authentication tokens match, control proceeds to step 1412. At step 1412, a flag is set (e.g., changed from a Boolean false to a Boolean true) or modified (e.g., changed from a Boolean true to a Boolean false value) in the memory of the medication delivery device to indicate that the medication delivery device has been connected to a user account. After step 1412, control proceeds to step 1410 where the process ends.

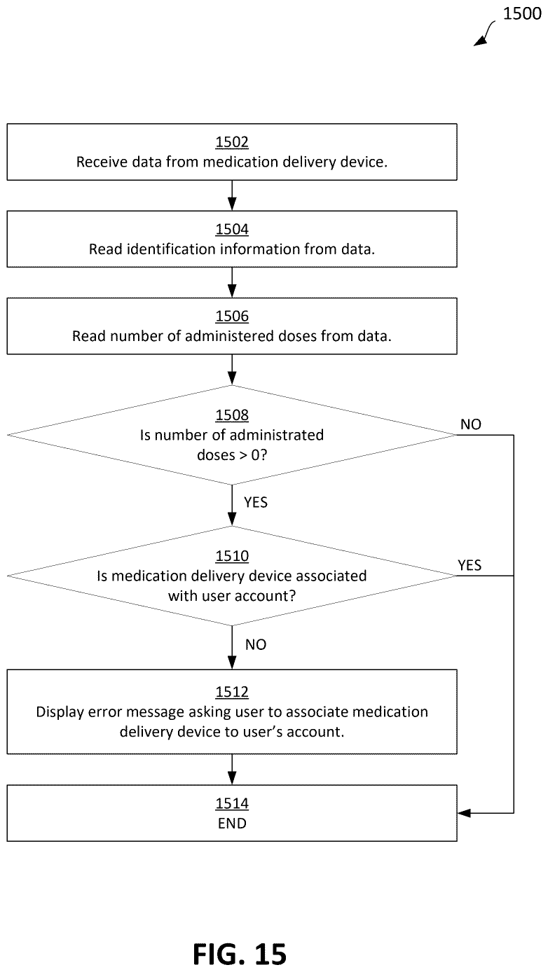

FIG. 15 is an exemplary flowchart of a method 1500 for receiving and processing an advertising packet from a medication delivery device. For the purposes of explication, method 1500 is described below as being implemented on a user's mobile device, such as computing device 1220. However, this is solely by way of example as method 1500 may be implemented on any suitable processor. For example, the step(s) may be performed by a laptop computer, a desktop computer, one or more servers, in a cloud computing environment, and/or in any other suitable way.

At step 1502, the mobile device receives an advertising packet and extracts information included therein. As described herein, such information may comprise the device identifier (e.g., 1202-1 . . . 1202-N) associated with the medication delivery device that transmitted the advertising packet. Such advertising packets may also contain at least the types of information previously described in relation to step 1311. In this exemplary embodiment, the advertising packets may include a dosage identifier that indicates the number of doses that the medication delivery device has delivered since it was first manufactured. For instance, such a dosage identifier may be initialized to zero when the delivery device is first manufactured, and every time the delivery dose delivers a dose (whether a priming dose or a therapeutic dose), the delivery device may be configured to increment the dosage identifier by 1. The dosage identifier, therefore, may be a number that indicates the number of doses that the delivery device has delivered in its lifetime. In some embodiments, the advertising packets may also contain a connection flag that indicates whether or not the delivery device has been connected to a user account, as previously described herein.

At step 1504, the mobile device determines whether the delivery device that transmitted the advertising packet is associated with the user account of the user currently logged into the mobile device. This may comprise consulting information 1226 in local memory 1224 to determine whether information 1226 contains the device identifier extracted from the advertising packet. If information 1226 does not include the extracted device identifier, that may mean the delivery device that transmitted the advertising packet has not previously been associated with the user account of the user currently logged into the mobile device. If that is the case, method 1500 proceeds to step 1506.

At step 1506, the mobile device determines whether the delivery device that transmitted the advertising packet is associated with any user account. If the advertising packet contains the aforementioned connection flag, this determination may be done simply by examining the connection flag to determine whether the delivery device has previously been associated with a user account. Alternatively, if the advertising packet does not contain the aforementioned connection flag, the mobile device may send a query to a remote server, e.g., server 1230. As described herein, the server 1230 may maintain a data store that indicates whether certain device identifiers have previously been connected to a user account. In response to the query from the mobile device, the server 1230 may return a message indicating whether the delivery device associated with the device identifier has previously been connected to a user account. If the delivery device had previously been connected with a user account, it may be that the mobile device has simply picked up an advertising packet transmitted by someone else's medication delivery device. Accordingly, method 1500 may proceed to step 1512 where it ends. If the delivery device has never previously been connected with a user account, method 1500 may proceed to step 1508.

At step 1508, the mobile device determines whether the number of administered doses fit certain error message criteria. In some embodiments, these criteria are used to determine whether the mobile device should present an error message to the user. Such criteria may include, for instance, evaluating whether the number of administered doses exceeds a predetermined minimum threshold, such as zero, one, or two doses. The number of administered doses may be determined by examining the previously discussed dosage identifier included in the advertising packet. If the number of administered doses exceeds zero, that may mean that, contrary to user instructions, the user has used the delivery device to deliver one or more doses of medication without first connecting the delivery device to a user account. Accordingly, if the number of administered doses is greater than zero or some other predetermined threshold (e.g., one, two doses), method 1500 may proceed to step 1510, where the mobile device presents the user with an error message.

Alternatively, or in addition, the criteria may include evaluating whether the number of administered doses is less than a predetermined maximum threshold, such as ten, fifteen, or twenty doses. If the number of administered doses is greater than this maximum threshold, the mobile device may conclude that the user is either (a) intentionally disregarding the instructions to first connect the delivery device to a user account before delivering a dose, and/or (b) the medication delivery device has previously been used by someone else and is transmitting from a nearby trash or sharps container. Accordingly, if the number of administered doses is less than the predetermined maximum threshold, method 1500 may proceed to step 1510. If the number of administered doses does not fit the error message criteria, e.g., it is less than the predetermined minimum threshold or greater than the predetermined maximum threshold, method 1500 may proceed to step 1512 where it ends.

At step 1510, the mobile device presents an error message to the user reminding the user to connect the medication delivery device to his/her user account before delivering a dose. The error message may provide the user an option to connect the delivery device to his/her user account. If the user selects this option, the user may be guided through method 1300 discussed above at least in relation to FIG. 13. In this scenario, the mobile device may guide the user through only some and not all of the steps described in method 1300—for example, the mobile device may implement only steps 1316, 1318, and 1320, since the earlier steps 1302, 1304, 1306, 1308, 1310, 1311, and 1312 may be rendered redundant.

In some embodiments, the mobile device and method 1500 may be configured to not prevent the user from using the delivery device without first connecting the device to his/her user account. Accordingly, in some embodiments, the user may simply dismiss the error message without connecting the delivery device to his/her user account. In some embodiments, the user can choose to delay connecting the medication delivery device to his/her user account. For example, the mobile device can include a “snooze” or “remind” feature that prompts the user at a later time to connect the delivery device to his/her user account. As another example, the mobile device can maintain a list of medication delivery devices that have been used but not connected to the user's account. The user can review such a list at their own leisure and/or the mobile device can be configured to periodically remind the user to connect any medication delivery devices in the list.

Steps 1504, 1506, 1508, and/or 1510 may be rearranged, altered, supplemented, or removed. For instance, in some embodiments, step 1508 may be removed entirely. Since, as described previously, the medication delivery device may be configured to refrain from transmitting advertising packets until it has delivered a first dose, the mere receipt of an advertising packet may indicate that the delivery device has already delivered at least one dose. In such cases, step 1508 may be omitted entirely and method 1500 may proceed directly from step 1506 to step 1510. Alternatively, step 1508 may be altered so that the number of administered doses is compared only to a predetermined maximum threshold and not a minimum threshold. In some embodiments, step 1506 may be omitted and method 1500 may proceed directly from step 1504 to step 1508. Omitting step 1506 may be advantageous for conserving computing, network, and/or battery resources because it relieves the mobile device from having to communicate with server 1230 every time it receives an advertising packet from a mobile device that does not match an entry in its local data table 1226. In yet other embodiments, the steps may be rearranged such that step 1508 is implemented before step 1504. Other alterations to steps 1504, 1506, 1508, and/or 1510 are also possible.

Returning to step 1504, if information 1226 includes the extracted device identifier, that may mean the delivery device that transmitted the advertising packet has already been associated with the user account of the user currently logged into the mobile device. If that is the case, method 1500 proceeds to step 1514.

At step 1514, the mobile device compares the number of doses (e.g., determined from reading the dosage identifier included in the advertising packet) with the number of doses stored in data table 1226. In other words, the mobile device determines whether the stored information in table 1226 comprises the dosage identifier included in the advertising packet received at step 1502. For instance, the mobile device consults the entry in table 1226 pertaining to the extracted device identifier to retrieve dosage identifier (1202-1c, . . . 1202-Nc, in FIG. 12). The dosage identifier saved into table 1226 indicates the last dose event that was recorded for the medication delivery device. If the dosage identifier retrieved from data table 1226 matches the dosage identifier extracted from the advertising packet, the mobile device may conclude that it has received information for every dose event that the delivery device has delivered. Accordingly, method 1500 may proceed to step 1512, where it ends.

If, however, the dosage identifier retrieved from data table 1226 is less than the dosage identifier extracted from the advertising packet, the mobile device may conclude that the delivery device has delivered one or more doses for which the mobile device has not yet received information. Accordingly, method 1500 may proceed to step 1516, where the mobile device sends a query to the medication delivery device. The query may include the device identifier for the medication delivery device such that the query does not elicit a response from other medication delivery devices that are not associated with said device identifier. The query may request that the delivery device provide information for each incremental dose that the delivery device has delivered since the last dose received by the mobile device, i.e., the dose corresponding to the dosage identifier saved in table 1226. For example, if the dosage identifier received from the medication delivery device indicates the delivery device has delivered four doses since it was first manufactured, but the dosage identifier stored in data table 1226 for the delivery device indicates only information relating to two doses has been received from this delivery device, then the mobile device requests information for doses three and four from the medication delivery device. In response, the delivery device may respond with dose information corresponding to the requested doses. Such dose information may include the amount of medication delivered with each dose, the time and/or date of each dose, and any other dose-specific information that may be recorded by the delivery device. Such dose information may also include information indicative of a status of the medication delivery device, such as information indicative of a power level of the particular medication delivery device, information indicative of an amount of medication held in the particular medication delivery device, information indicative of a type of medication held in the particular medication delivery device, information indicative of a time the particular medication delivery device transitioned to a connected state, and/or information indicative of a time the particular medication delivery device was activated. Such dose information may optionally be encrypted using the aforementioned encryption key such that it can be decrypted by the mobile device (which received the encryption key from the server 1230, as described above in connection with method 1300), but cannot be decrypted by any other eavesdropping device. After the mobile device has retrieved (and optionally, decrypted) information relating to any such incremental doses, method 1500 proceeds to step 1512, where it ends.

It should be appreciated that any combination of steps may be performed as part of method 1500. Method 1500 may include additional or fewer steps than those shown in FIG. 15. As should be appreciated from the foregoing, the techniques described herein may be used to retrieve information from a medication delivery device without establishing a formal paired session, e.g., under the Bluetooth or Bluetooth Low Energy (BLE) protocol. Rather, to receive information associated with a medication delivery device, the user may simply place the medication delivery device in range of the mobile device. The information may be included in an advertising packet transmitted by the medication delivery device and received by the computing device, or it may be included in a response to a query transmitted by the computing device.

Method 1500 describes a method by which information relating to doses may be automatically “pushed” from the delivery device to the mobile device without user intervention. As discussed above, for instance, the automatic transmission of advertising packets from the delivery devices causes the mobile device to automatically recognize when it needs to request incremental dose information, and to automatically retrieve such information as needed. This process may be done seamlessly and invisibly to the user and may not require any user intervention.

In some embodiments, information may also be “pulled” from the delivery device by the mobile device when requested by the user. For instance, the user may wish to query a particular delivery device to determine a status of said delivery device, such as its battery level, expiration date, remaining stock of medication, activation date, date and/or time of last dose, and/or a type of medication stored therein. Such a query may be initiated by the user and not by the medication delivery device. However, the inventors have recognized that there may be challenges associated with pulling information from a particular medication delivery device. For example, there may be situations where more than one medication delivery device is in range of the mobile device. Accordingly, it may be challenging to identify a particular medication delivery device that a user is interested in pulling information from (e.g., the particular device that the user may be holding in his/her hand, as opposed to another device in the user's backpack or purse). Additionally or alternatively, in the case where the mobile device transmits a query to a medication delivery device, it may be challenging to identify the particular medication device being queried. In both cases, the mobile device may receive information from multiple medication delivery devices. As a result, it is unclear which information has been received from the particular medication delivery device and should be presented to the user.

While the user could physically separate the mobile device and the additional medication delivery devices such that they are out-of-range of one another, this would place an unnecessary burden on the user. For example, if the user lives in a small home, establishing a sufficient physical separation may require the user to leave his or her home. Additionally or alternatively, if the user is attempting to query a medication delivery device in a public setting, a hospital, a nursing home, and/or the like, the user may not be able to control the separation between their computing device and medication delivery devices belonging to other users.

Accordingly, the inventors have developed techniques that address the above-described challenges associated with pulling information from a particular medication delivery device.

In some embodiments, the techniques include (a) receiving a set of advertising packets, including at least one advertising packet from one or more medication delivery devices, (b) determining a number of unique identifiers included in the set of advertising packets to determine a number of transmitting devices, (c) determining, based on the number of transmitting medication delivery devices, whether to prompt the user to provide identification information for the particular medication delivery device; and (d) presenting the information from the particular medication delivery device to the user.

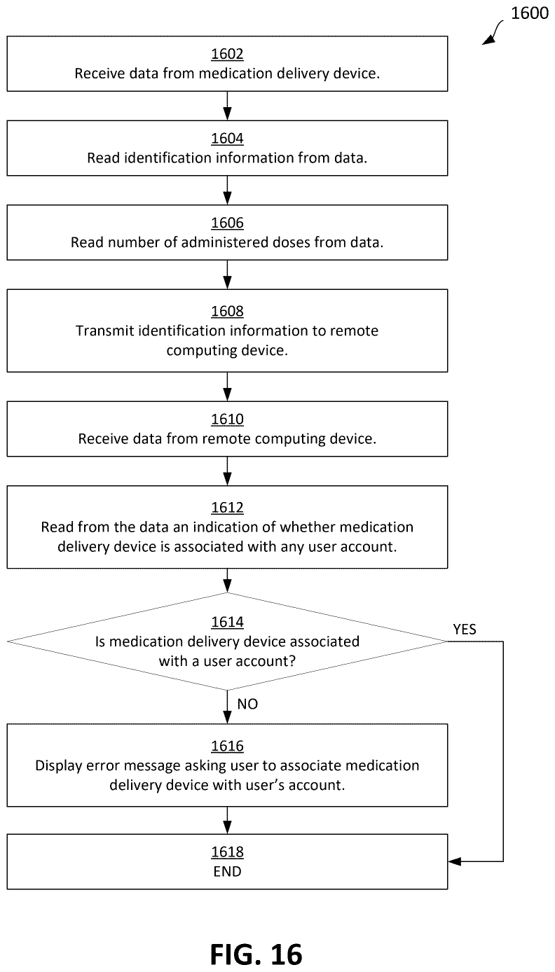

FIG. 16 is a flowchart showing an exemplary method 1600 for pulling and presenting information from a particular medication delivery device, according to some embodiments. For the purposes of explication, method 1600 is described below as being implemented on a user's mobile device, such as computing device 1220. However, this is solely by way of example as method 1600 may be implemented on any suitable processor. For example, the step(s) may be performed by a laptop computer, a desktop computer, one or more servers, in a cloud computing environment, and/or in any other suitable way.

At step 1602, the mobile device receives input requesting information from a particular medication delivery device. In some embodiments, the mobile device receives the input in response to prompting the user for the input. For example, the mobile device may generate a graphical user interface (GUI) including one or more selectable elements. A selectable element may include an option relating to requesting information from a medication delivery device. For example, the GUI may include a list of selectable elements corresponding to a list of medication delivery devices. Additionally or alternatively, the GUI may include a list of selectable elements corresponding to types of information (e.g., status information, dosage information, etc.) that can be requested from a particular medication delivery device. In some embodiments, the mobile device receives the input in response to a user interacting with such selectable elements. Additionally or alternatively, in some embodiments, receiving the input includes receiving the input from a source other than the user. For example, software executed by the mobile device may include instructions for requesting information from the particular medication delivery device at periodic intervals (e.g., request information from the particular medication delivery device every 12 hours) or aperiodic intervals. While various examples of receiving input for requesting information from a particular medication delivery device have been described, it should be appreciated that input may be received in any other suitable manner, as aspects of the technology are not limited in this respect.

At step 1603, the mobile device monitors for any received advertising packets. If no advertising packets are received (e.g., within a predetermined time period of step 1602), the method 1600 proceeds to the error condition of step 1605. For example, the medication delivery device may be too far away for the mobile device to receive the advertising packets. As another example, the medication delivery device may be damaged and/or its battery may be low or dead, such that the medication delivery device is not transmitting (or is unable to properly transmit) advertising packets. As part of step 1605, the mobile device may indicate to the user that no advertising packets have been received for the medication delivery device identified at step 1602 (e.g., and may provide troubleshooting information). Otherwise, if at least one advertising packet is detected at step 1603, the method 1600 proceeds to step 1604.

At step 1604, the mobile device receives a set of advertising packets. The set of advertising packets includes at least one (e.g., 1, 2, . . . N) advertising packet from one or more (e.g., 1, 2, . . . M) medication delivery devices. As a non-limiting example, this may include receiving one advertising packet from a first medication delivery device, three advertising packets from a second medication delivery device, and two advertising packets from a third medication delivery device. Additionally or alternatively, this may include receiving several advertising packets from a single medication delivery device.

In some embodiments, each of the advertising packets includes an identifier associated with a respective medication delivery device. The identifier may be used to distinguish between advertising packets received from different medication delivery devices. For example, advertising packets received from a first medication delivery device may include a first identifier and advertising packets received from a second medication delivery device may include a second identifier different from the first identifier. The identifier may include a unique serial number, sequence of characters, or any other suitable identifier that is unique to a particular medication delivery device and that can be included in an advertising packet, as aspects described herein are not limited in this respect.

At step 1606, the mobile device determines a number of unique identifiers included in the set of advertising packets to determine a number of transmitting medication delivery devices. In some embodiments, the number of unique identifiers is equivalent to the number of transmitting medication delivery devices. For example, if the set of advertising packets includes five different identifiers, this indicates that there are five transmitting medication delivery devices. If the set of advertising packets includes only one identifier, this indicates that there is only one transmitting medication delivery device, even when the set of advertising packets includes multiple advertising packets.

At step 1608, the mobile device determines whether to prompt the user to provide identification information. In some embodiments, the identification information includes information unique to the particular medication delivery device. For example, the identification information may include information indicative of the identifier included in the advertising packets transmitted by the particular medication delivery device. The identification information may include a unique serial number, sequence of characters, barcode, or any other suitable identification information. In some embodiments, the identification information is displayed on the particular medication delivery device (e.g., on a label or the housing of the medication delivery device). In yet other embodiments, the identification information may comprise a user-assigned nickname or personalized name for the particular medication delivery device.

In some embodiments, the mobile device determines whether to prompt the user to provide the information based on the number of transmitting devices, determined at step 1606. For example, if there is only one transmitting medication delivery device, there is no need to distinguish between different medication delivery devices. Accordingly, there may be no reason to burden the user by prompting the user to provide the identification information. By contrast, if there are multiple transmitting devices, identification information may be useful in distinguishing between them. In this case, the mobile device may prompt the user to provide the identification information.

If, at step 1608, the mobile device determines not to prompt the user to provide identification information, then method 1600 proceeds to step 1610. At step 1610, the mobile device determines whether the information (e.g., the information requested at step 1602) is included in one or more of the advertising packets received at step 1604.

If the mobile device determines at step 1610, that the information is included in an advertising packet, then method 1600 proceeds to step 1612. At step 1612, the mobile device presents the information included in the advertising packet without prompting the user to provide identification information. In other words, if there is only one transmitting device and the requested information is included in the advertising packet, then the mobile device may automatically present the requested information.

In some embodiments, the information may be presented in any suitable manner as aspects of the technology described herein are not limited in this respect. For example, the information may be presented through a GUI. The GUI may include text, graphics, or any other suitable elements for presenting the information. Additionally or alternatively, the mobile device may store the information in memory (e.g., memory 1224), transmit the information to a server (e.g., server 1230), transmit the information to another computing device, or use the information in any other suitable way, as aspects of the technology described herein are not limited in this respect. For example, the information may be processed to determine a dose schedule, reviewed by a healthcare provide, or used for any other suitable application.

If, at step 1610, the mobile device determines that the information is not included in the advertising packet(s), the method 1600 proceeds to step 1614. At step 1614, the mobile device transmits, based on the identifier included in each advertising packet, a query configured to provoke a response from the particular medication delivery device. For example, since the advertising packets are all received from the same medication delivery, each advertising packet will include the same identifier. Thus, in some embodiments, the mobile device can use the identifier included in the advertising packets to transmit the query and provoke a response from the particular medication delivery device with some and/or all of the information. All other medication delivery devices may be configured to not respond to any queries that includes an identifier that does not match their own identifiers.

At step 1616, the mobile device receives a response from the particular medication delivery device and presents the information included in the response. The mobile device may present the information using any suitable techniques such as, for example, those described herein including at least with respect to step 1612.

In some embodiments, the mobile device determines, at step 1608, to prompt the user to provide identification information. For example, the process may prompt the user to provide identification information if there is more than one transmitting medication delivery device. In some embodiments, the mobile device prompts the user by generating a GUI that is presented to the user and includes text and/or other visual indications requesting the information. Additionally or alternatively, the mobile device may generate audio output that includes a verbal and/or other audible indications requesting the information. However, it should be appreciated that aspects of the technology described herein are not limited in this respect, and the mobile device may prompt the user for the identification information using any other suitable techniques.

At step 1620, after prompting the user to provide the identification information, the mobile device receives the identification information. The mobile device may receive the identification information as user input through a user interface associated with the mobile device, in response to the scanning of a barcode (e.g., QR code), from a server, from memory, or in any other suitable manner, as aspects of the technology are not limited in this manner.

At step 1621, the mobile device checks whether the medication delivery device is registered to the user. If the device is registered to the user, the method 1600 proceeds to step 1622. If not, the method 1600 proceeds to the error condition of step 1623. Step 1623 may occur, for example, if the medication delivery device is registered to another user. As another example, the medication delivery device may not be registered to any user. If the medication delivery device is not registered, the mobile device can indicate as such to the user and/or guide the user through the registration process as discussed herein.

At step 1622, the mobile device determines whether the information (e.g., the information requested at step 1602) is included in advertising packet(s) received at step 1604.

If, at step 1622, the mobile device determines that the information is included in the advertising packets, then the method proceeds to step 1624. At step 1624, the mobile device identifies the advertising packet(s) that were received from the particular medication delivery device. In some embodiments, the mobile device uses the identification information received at step 1620 to identify the advertising packet(s) from the particular medication delivery device. For example, the identification information may include the identifier associated with the particular medication delivery device or information indicative of the identifier. Accordingly, the mobile device may compare the identification information to the identifiers included in the advertising packets. The mobile device may then identify advertising packet(s) including an identifier that matches the identifier indicated by the identification information.

At step 1626, the mobile device presents the information included in the identified advertising packet(s). The mobile device may present the information using any suitable techniques such as, for example, those described herein including at least with respect to step 1612.

If, at step 1622, the mobile device determines that the information is not included in the advertising packet(s), then method 1600 proceeds to step 1628. At step 1628, the mobile device transmits, based on the identification information, a query configured to provoke a response from the particular medication delivery device. Since the advertising packet(s) were received from multiple different medication delivery devices, they will include multiple different identifiers. Since it is unclear which of the multiple identifiers is associated with the particular medication delivery device, the identifiers cannot be used to query the particular medication delivery device, as they are in step 1614 of method 1600. Rather, the identification information is used to identify and query the particular medication delivery device.

At step 1630, the mobile device presents the information included in the response from particular medication delivery device. The mobile device may present the information using any suitable techniques such as, for example, those described herein including at least with respect to step 1612.

It should be appreciated that any combination of steps may be performed as part of method 1600. Method 1600 may include additional or fewer steps than those shown in FIG. 16, since the steps can be rearranged, altered, supplemented, or removed. As a non-limiting example, in some embodiments, step 1608 can occur before step 1604.

As described herein, connecting medication delivery devices over time can result in an undesirably large number of devices in an associated device list. Such a long list can be cumbersome to navigate by a user and/or consume unnecessary computing resources. The inventors have recognized that it is desirable to retire a medication delivery device under certain circumstances (e.g., when the device is no longer in-use and/or usable by the user). The inventors have therefore developed techniques for retiring a medication delivery device. In some embodiments, the medication delivery device is retired by deleting the medication delivery device from a list of devices and/or moving the medication delivery device's entry from the list to a different list.

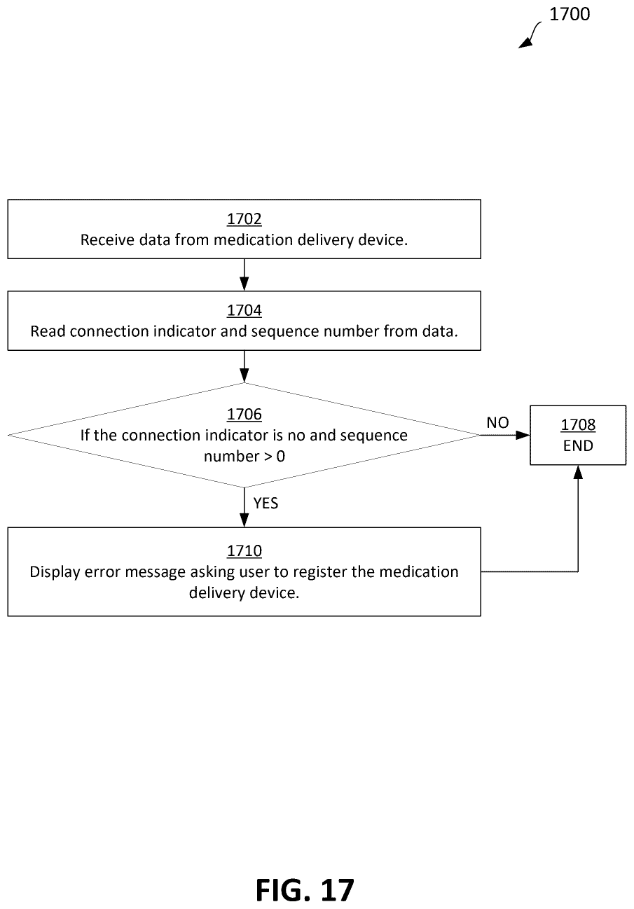

FIG. 17 is a flowchart of an exemplary method 1700 (e.g., executed at a user's mobile device) for determining whether to retire a medication delivery device. At step 1701, the method 1700 monitors for data and/or messages from a medication delivery device. If the mobile device does not receive any data from the medication delivery device (e.g., within a predetermined time period), the method 1700 can proceed to step 1706. Otherwise, the method proceeds to step 1702.

At step 1702, data associated with a medication delivery device is received. At step 1704, a determination is made as to whether to retire the medication delivery device based on the received data. If it is determined at step 1704 to retire the medication delivery device, the medication delivery device is removed from a list of active medication delivery devices in step 1706. Next, at step 1708, the medication delivery device is optionally added to a separate list, such as a list of retired medication delivery devices. If, on the other hand, it is determined at step 1704 to not retire the medication delivery device, control returns to step 1702 to await more data from the medication delivery device.

Referring to steps 1702 and 1704, various data can be checked to determine whether to retire a medication delivery device, such as a number of days of use, a low battery indication, and/or an indication that the medication delivery device is running low of medication. In some embodiments, the assessed data comprises a time of use of the medication delivery device. In some embodiments, determining to retire the medication delivery device comprises determining the time of use (e.g., the date and time) is greater than a threshold time. In some embodiments, the time of use of the mediation delivery device is determined based on a difference between a current time and one of (a) a time when the medication delivery device was first associated with the user account, and (b) a time when the medication delivery device delivered a first dose. In some embodiments, the threshold time comprises a number of days (e.g., greater than three weeks/twenty-one days, four weeks/twenty-eight days, etc.). In some embodiments, the assessed data comprises data indicative of a battery level of the medication delivery device. In some embodiments, determining to retire the medication delivery device comprises determining the battery level indicator is below a predetermined threshold.

In some embodiments, the assessed data comprises data indicative of a remaining amount of medicine of the medication delivery device. In some embodiments, determining to retire the medication delivery device comprises determining, based on the remaining amount of medicine, the medication delivery device has no remaining medicine.

In some embodiments, a user can be queried to check and/or confirm whether to retire a medication delivery device. For example, in some embodiments determining to retire the medication delivery device comprises (i) requesting input from a user to provide data indicating whether the medication delivery device should be retired, and (ii) determining, responsive to the received data, to retire the mediation delivery device.

FIG. 18 is a flowchart of an alternative exemplary method 1800 for retiring a medication delivery device that has little or no medicine, a low battery, and/or is otherwise unusable, according to some embodiments.

At step 1802, a message is received from a medication delivery device at the user's mobile device. At step 1804, a serial number (e.g., device identifier) is read from the message at the user's mobile device. At step 1806, a determination is made as to whether the serial number for the medication delivery device is stored in a table (e.g., table 1226) on the user's mobile device. If it is determined that the serial number for the medication delivery device is stored in the table on the user's mobile device at step 1806, control proceeds to step 1808. At step 1808, the date that the medication delivery device was connected to the user's account (e.g., using the method described in relation to FIG. 13) is read from the table in the memory of the user's mobile device. At step 1810, a determination is made as to whether the period of time that has elapsed since the medication delivery device was first connected to the user's account is greater than a predetermined threshold. In some embodiments, the predetermined threshold is a time period that the user is expected to use the medicine in the medication delivery device. In another embodiment, the predetermined threshold is a time period after which the medicine will expire (e.g., no longer have the desired efficacy). If it is determined in step 1810 that the period of time that has elapsed since the medication delivery device was first connected to the user's account is greater than the predetermined threshold, control proceeds to step 1818.

At step 1818, input is requested from the user as to whether to retire the medication delivery device. At step 1820, the user's mobile device receives the response from the user. At step 1822, a determination is made at the user's mobile device whether the user responded that the medication delivery device should be retired. If it is determined at step 1822 that the response from the user indicates that the medication delivery device should be retired, control proceeds to step 1824. At step 1824, the user's mobile device retires the medication delivery device. In some embodiments, the medication delivery device is retired by deleting its entry in the table (e.g., table 1226) of medication delivery devices in the memory of the user's mobile device. In other embodiments, the medication delivery device is retired by moving its entry in the table of medication delivery devices to another table of retired medication delivery devices. In some embodiments, the entries in the table of retired medication delivery devices are deleted after a predetermined time period (e.g., 30 days). If it is determined at step 1822 that the response from the user indicates that the medication delivery device should not be retired, control proceeds to step 1826 where the process ends.

If it is determined at step 1810 that the period of time that has elapsed since the medication delivery device was first connected to the user is not greater than the predetermined threshold, control proceeds to step 1812. At step 1812, a status request is sent from the user's mobile delivery device to the medication delivery device. In some embodiments, the medication delivery device sends a response to the status request to the user's mobile delivery device. In some embodiments, the response includes an indicator of whether the battery in the medication delivery device is low, an indicator of whether the amount of medicine remaining in the medication delivery device is below a predetermined threshold, and/or an indicator that there is no medicine remaining in the medication delivery device. At step 1813, the status response from the medication delivery device is received at the user's mobile device. At step 1814, a determination is made as to whether the status response from the medication delivery device indicates that there is no medicine remaining in the medication delivery device. If a determination is made in step 1814 that the response from the medication delivery device indicates that it does not hold any medicine, control proceeds to step 1824, where the medication delivery device is retired as explained above.

If a determination is made in step 1814 that the response from the medication delivery device indicates that it does hold some medicine, control proceeds to step 1816. At step 1816, a determination is made as to whether the response from the medication delivery device indicates that its battery is low and/or the amount of remaining medication within it is below the predetermined threshold. If a determination is made in step 1816 that the response from the medication delivery device indicates that either its battery is low or that the amount of medication within it is below the predetermined threshold, then control proceeds to step 1818, where a message is sent to the user asking if the medication delivery device should be retired as explained above. If a determination is made at step 1814 that the response from the medication delivery device indicates that its battery is not low and that the amount of remaining medicine is not below the predetermined threshold, control proceeds to step 1826 where the process ends. If a determination is made at step 1806 that the serial number read from the message from the medication delivery device at step 1804 is not in the table of medication delivery devices in the memory of the user's mobile device, control proceeds to step 1826 where the process ends. Alternatively or in addition, control may proceed to method 1500 discussed herein at least in relation to FIG. 15.

While method 1800 includes various determinations as discussed above, including checking the period of time of use of the medication delivery device (e.g., step 1810), checking whether the medication delivery device is empty (e.g., step 1814), checking whether the medication delivery device has a low battery and/or low medicine (e.g., step 1816), and confirming with a user whether to retire the medication delivery device (e.g., step 1822), it should be appreciated that each of these various determinations can be made on its own and/or in combination with one or more other determinations, and that the techniques are not limited to performing all of the exemplary determinations shown in FIG. 18. While not shown in method 1800, it should be appreciated that method 1800 can additionally or alternatively include monitoring for message(s) from a medication delivery device (e.g., as discussed in conjunction with step 1701 of FIG. 17). If no data or messages are received (e.g., within a predetermined period), the method 1800 can proceed to step 1824 accordingly to retire the medication delivery device.

In some embodiments, one or more medication delivery devices may be subject to a recall. If recalled, it can be desirable to disable and/or remotely modify one or more aspects of such medication delivery devices. For example, it can be desirable to prevent use of the medication delivery device and/or to cease any communications with such a medication delivery device. In some embodiments, the techniques described herein provide for a computing device, such as a user's mobile device, to receive information regarding recalled medication devices. In some embodiments, the mobile device can be configured to terminate any active communications with the recalled devices. For example, any communications between the mobile device and the medication delivery device can be terminated (e.g., for medication delivery devices registered to the user's account and/or for medication delivery devices in active communication with the mobile device). In some embodiments, the mobile device can be configured to prevent any new or future communications with recalled devices. For example, the mobile device may prevent the user from registering recalled devices with the user's account. As a further example, the mobile device can store a list of recalled devices that can be checked prior to initiating communication with a new device. In some embodiments, the user may be presented with information on a recall and/or medication delivery device(s) that are subject to recall.

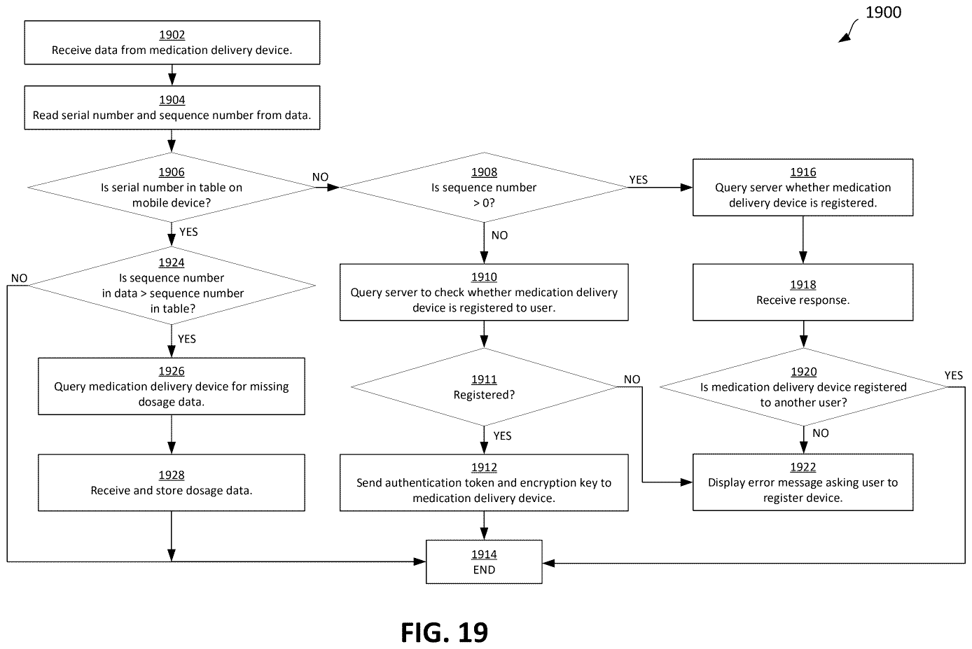

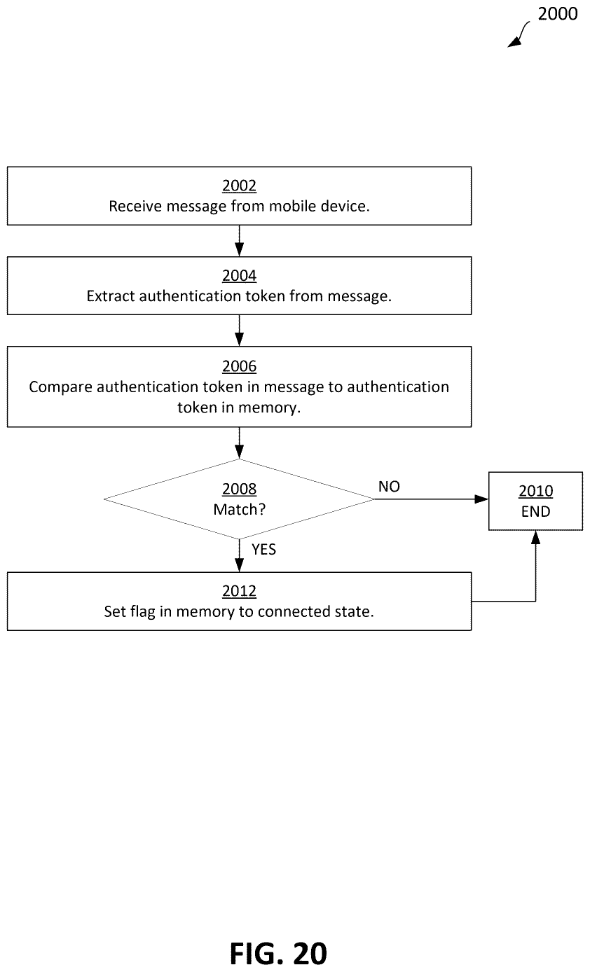

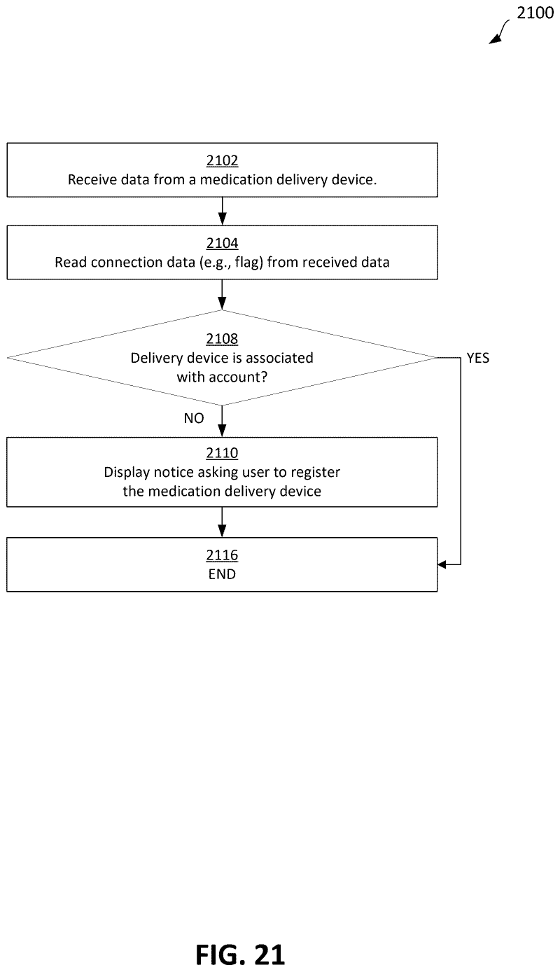

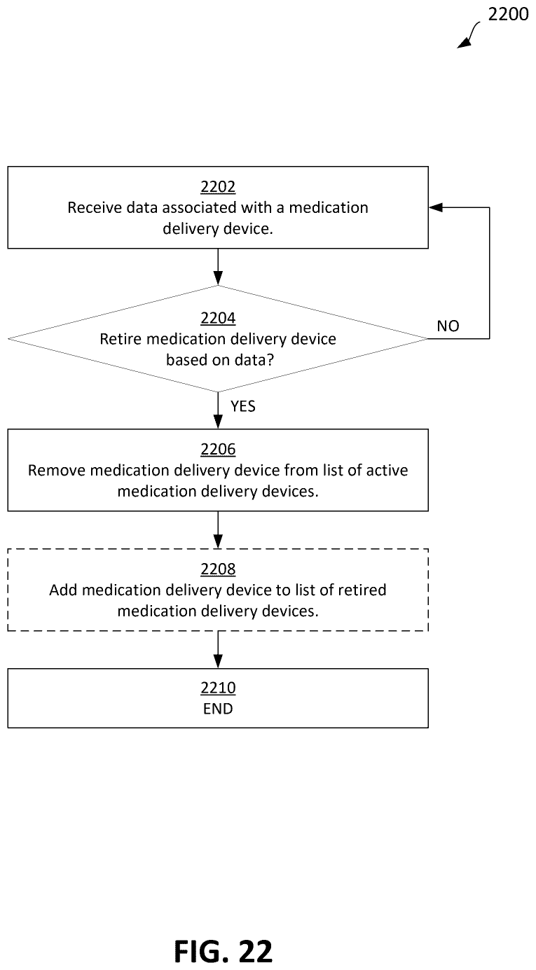

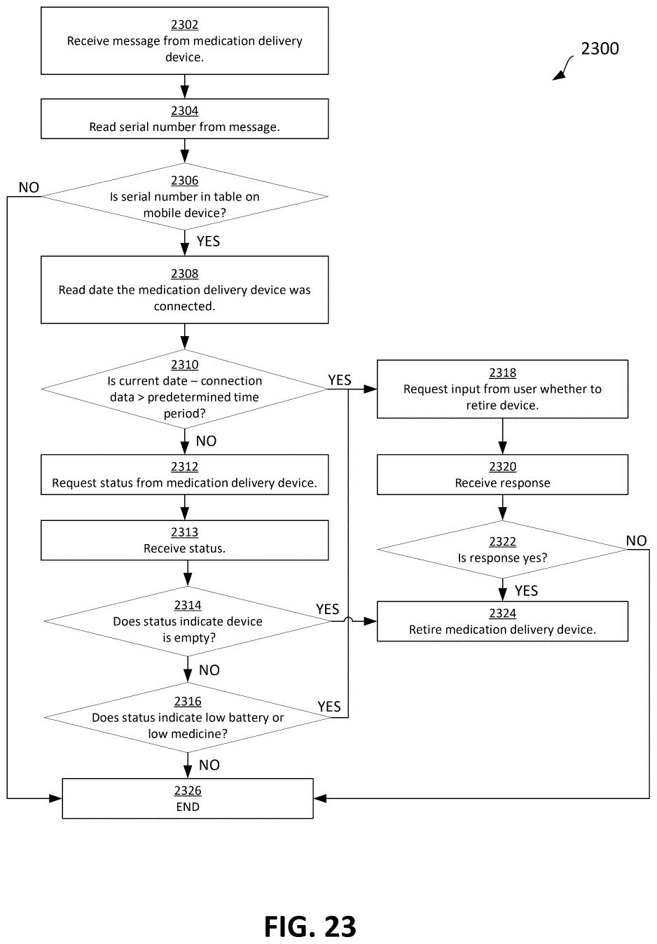

FIG. 19 is a flowchart showing an exemplary method 1900 for preventing communications with a medication delivery device subject to recall, according to some embodiments. For the purposes of explication, method 1900 is described below as being implemented on a user's mobile device, such as computing device 1220. However, this is solely by way of example as method 1900 may be implemented on any suitable processor. For example, the step(s) may be performed by a laptop computer, a desktop computer, one or more servers, in a cloud computing environment, and/or in any other suitable way.

At step 1902, the mobile device receives data indicating medication delivery device(s) subject to recall. For example, in the event of a recall of a specific set (e.g., a certain batch) of medication delivery devices, it may be possible to send a list of identifying information, such as a list of serial numbers, corresponding to the recalled devices to all mobile devices. The information can be received from, for example, a remote computing device or server, such as server 1230. As described herein, the recalled medication delivery devices can be of various types. For example, the medication delivery device can be a smart pen with integrated components to track and report information, such as dosing information, wirelessly to a receiving device. As another example, the medication delivery device can be a component, such as a smart module, that can be mounted on a medication delivery device to track and report dose information.

At step 1904, the mobile device determines whether it is in communication with any of the medication delivery device(s) subject to recall. If so, the method 1900 proceeds to step 1906, and the mobile device immediately ceases to accept communications and/or data (e.g., dosing related data) from the recalled devices. This can include, for example, terminating any active communications with such devices.

The method proceeds to step 1908 and stores data, such as the device serial numbers, so that the mobile device can be prevented from establishing any communications with such device(s). The information can be stored, for example, in a list or table of medication delivery devices that are recalled and/or otherwise blacklisted from communication with the mobile device. In some embodiments, the owner or user of such recalled devices may still be able to use the devices, but is prevented from using any connected features of the devices, such as dose logging, medication delivery device querying, etc.

Optionally, the method 1900 can proceed to step 1910 and the user can be notified of the recall. In some embodiments, the user can be notified that there was a recall and/or provided with general information regarding the recall (e.g., why the devices were recalled). In some embodiments, the user can additionally or alternatively be notified of the specific medication delivery devices subject to recall (e.g., by product name and/or by serial number). In some embodiments, the user can be notified that one or more of the devices registered to the user's account were subject to recall.

It should be appreciated that any combination of steps may be performed as part of method 1900. Method 1900 may include additional or fewer steps than those shown in FIG. 19, since the steps can be rearranged, altered, supplemented, or removed. For example, in some embodiments, step 1908 and/or step 1910 can occur before step 1906.

While some exemplary embodiments discussed herein (e.g., in conjunction with FIG. 19) prevent communications with a medication delivery device via the mobile device, it should be appreciated that other techniques can be used in addition to, or instead of, such approaches. In some embodiments, for example, the medication delivery device itself can be modified or adjusted accordingly. As an example, the mobile device (e.g., acting on instructions from a remote server), can modify, patch, and/or update the software or firmware of the medication delivery device. For example, the software or firmware can be modified to prevent communications from the medication delivery device. As another example, the medication delivery device can be disabled so that the medication delivery device cannot deliver medication. As a result, the techniques described herein can be used to disable a recalled medication delivery device by modifying the medication delivery device (e.g., to prevent communications, to modify the device functionality, etc.) and/or by having the mobile device no longer communicate with the recalled medication delivery device.

It should be appreciated that various types of medication delivery devices and electronics configurations can be used in conjunction with the techniques described herein. In some embodiments, the medication delivery device can be a fully integrated drug-delivery device that can sense and report dosage information to a mobile device. For example, as discussed above in conjunction with FIG. 5, many, if not all, of the components of the electronics assembly may be contained within and permanently integrated with the dose button. In some embodiments, as also discussed in conjunction with FIG. 5, some or all of the electronics assembly may be detachably coupled with the medication delivery device. For example, the electronics assembly can be provided as a module that can be removably attached to the actuator assembly of the medication delivery device. In such examples, the medication delivery device can be, for example, a mechanical pen that injects the drug, and the module can be detachably coupled with the delivery device to sense dispensed dose(s) and report such sensed dose information to a mobile device (e.g., via Bluetooth).

Techniques operating according to the principles described herein may be implemented in any suitable manner. The processing and decision blocks of the flow charts above represent steps and acts that may be included in algorithms that carry out these various processes. Algorithms derived from these processes may be implemented as software integrated with and directing the operation of one or more single-or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

Further, some techniques described above comprise acts of storing information (e.g., data and/or instructions) in certain ways for use by these techniques. In some implementations of these techniques-such as implementations where the techniques are implemented as computer-executable instructions-the information may be encoded on a computer-readable storage media. Where specific structures are described herein as advantageous formats in which to store this information, these structures may be used to impart a physical organization of the information when encoded on the storage medium. These advantageous structures may then provide functionality to the storage medium by affecting operations of one or more processors interacting with the information; for example, by increasing the efficiency of computer operations performed by the processor(s).

In some, but not all, implementations in which the techniques may be embodied as computer-executable instructions, these instructions may be executed on one or more suitable computing device(s) operating in any suitable computer system, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

A computing device may comprise at least one processor, a network adapter, and computer-readable storage media. A computing device may be, for example, a desktop or laptop personal computer, a personal digital assistant (PDA), a smart mobile phone, a server, or any other suitable computing device. A network adapter may be any suitable hardware and/or software to enable the computing device to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media may be adapted to store data to be processed and/or instructions to be executed by processor. The processor enables processing of data and execution of instructions. The data and instructions may be stored on the computer-readable storage media.

A computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B,. or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

Various aspects are described in this disclosure, which include, but are not limited to, the following aspects:
1. A method for presenting information from a particular medication delivery device to a user, the method comprising: receiving a set of advertising packets, the set of advertising packets comprising at least one advertising packet from one or more medication delivery devices, the one or more medication delivery devices including the particular medication delivery device, wherein each advertising packet includes an identifier associated with a respective medication delivery device of the one or more medication delivery devices; determining a number of unique identifiers included in the set of advertising packets to determine a number of transmitting medication delivery devices; determining, based on the number of transmitting medication delivery devices, whether to prompt the user to provide identification information for the particular medication delivery device; and presenting the information from the particular medication delivery device to the user.
2. The method of aspect 1, further comprising: upon determining that the number of transmitting medication delivery devices includes more than one medication delivery device, prompting the user to provide the identification information for the particular medication delivery device; receiving the identification information; and identifying, using the identification information, an advertising packet of the at least one advertising packet, the advertising packet having been received from the particular medication delivery device, wherein presenting the information from the particular medication delivery device to the user comprises presenting information included in the identified advertising packet.
3. The method of aspect 1, further comprising: upon determining that the number of transmitting medication delivery devices includes more than one medication delivery device, prompting the user to provide the identification information for the particular medication delivery device; receiving the identification information; transmitting, based on the identification information, a query configured to provoke a response from the particular medication delivery device; and receiving the response from the particular medication delivery device; wherein presenting the information from the particular medication delivery device to the user comprises presenting information included in the response from the particular medication delivery device.
4. The method of aspect 1, further comprising: upon determining that the number of transmitting medication delivery devices does not include more than one medication delivery device, presenting the information from the particular medication delivery device without prompting the user to provide the identification information for the particular medication delivery device, wherein presenting the information from the particular medication delivery device comprises presenting information included in an advertising packet of the at least one advertising packet.
5. The method of aspect 1, further comprising: upon determining that the number of transmitting medication delivery devices does not include more than one medication delivery device, transmitting, based on the identifier for each advertising packet, a query configured to provoke a response from the particular medication delivery device without prompting the user to provide the identification information for the particular medication delivery device; and receiving the response from the particular medication delivery device; wherein presenting the information from the particular medication delivery device to the user comprises presenting information included in the response from the particular medication delivery device.
6. The method of any of aspects 1-5 wherein the information from the particular medication delivery device comprises information indicative of one or more of: a status of the particular medication delivery device, a time associated with a dose of medication delivered using the particular medication delivery device, or a dosage amount associated with the dose of medication.
7. The method of aspect 6, wherein the information indicative of the status of the particular medication delivery device comprises one or more of: information indicative of a power level of the particular medication delivery device, information indicative of an amount of medication held in the particular medication delivery device, information indicative of a type of medication held in the particular medication delivery device, information indicative of a time the particular medication delivery device transitioned to a connected state, or information indicative of a time the particular medication delivery device was activated.
8. The method of any of aspects 1-7, wherein prompting the user to provide the identification information comprises prompting the user to perform one or more of: scanning a barcode associated with the medication delivery device or providing input through a user interface.
9. The method of any of aspects 1-8, further comprising receiving input requesting the information from the particular medication delivery device, wherein determining whether the number of transmitting medication delivery devices includes more than one medication delivery device is responsive to receiving the input.
10. A non-transitory computer-readable media comprising instructions that, when executed by one or more processors on a computing device, are operable to cause the one or more processors to execute the method of any of aspects 1-9.
11. A system comprising a memory storing instructions, and a processor configured to execute the instructions to perform the method of any of aspects 1-9.
12. A method for wirelessly connecting to a medication delivery device, the method comprising: receiving a device identifier associated with the medication delivery device at a computing device associated with a user; determining, based on the device identifier, whether the medication delivery device is associated with a different user other than the user; upon determining that the medication delivery device is not associated with the different user, storing, at the computing device, information associated with the medication delivery device, the information comprising an encryption key; receiving, at the computing device, encrypted information from the medication delivery device; and using the encryption key to decrypt the encrypted information received from the medication delivery device.
13. The method of aspect 12, wherein the stored information associated with the medication delivery device further comprises an authentication token, the method further comprising: determining whether another advertising packet was previously-received by the computing device from the medication delivery device; and upon determining that another advertising packet was not previously-received from the medication delivery device, transmitting the authentication token from the computing device to the medication delivery device.
14. The method of aspect 13, wherein transmitting the authentication token to the medication delivery device causes the medication delivery device to transition to a connected state.
15. The method of any of aspects 13-14, wherein determining whether another advertising packet was previously-received from the medication delivery device comprises analyzing a list of medication delivery devices associated with the user to determine whether any medication delivery device, in the list of medication delivery devices, is associated with the device identifier.
16. The method of any of aspects 12-15, further comprising: receiving a dosage identifier associated with the medication delivery device, the dosage identifier being indicative of a dose event, wherein the dose event is associated with a dosage amount and a unique dosage time.
17. The method of aspect 16, further comprising: determining, based on the dosage identifier, whether the stored information associated with the medication delivery device includes the dosage identifier; and upon determining that the stored information does not include the dosage identifier, requesting, from the medication delivery device, the encrypted information, wherein the encrypted information comprises one or more of the dosage amount or the unique dosage time associated with the dose event.
18. The method of any of aspects 12-17, wherein the encrypted information further comprises status information, the status information comprising one or more of: information indicative of a power level of the medication delivery device, information indicative of an amount of medication held in the medication delivery device, information indicative of a type of medication held in the medication delivery device, information indicative of a time the medication delivery device transitioned to a connected state, or information indicative of a time the medication delivery device was activated.
19. The method of any of aspects 12-18, wherein the device identifier is received through one or more of (i) scanning a barcode associated with the medication delivery device. (ii) providing input through a user interface, or (iii) receiving an advertising packet.
20. The method of any of aspects 12-19, further comprising: receiving user identification information associated with the user prior to receiving the device identifier, wherein storing the information associated with the medication delivery device comprises storing the information in connection with the user identification information.
21. The method of any of aspects 12-20, wherein determining whether the medication delivery device is associated with the different user other than the user comprises transmitting the device identifier to a server, and wherein the method further comprises, after transmitting the device identifier to the server, receiving, from the server the information associated with the medication delivery device.
22. A non-transitory computer-readable media comprising instructions that, when executed by one or more processors on a computing device, are operable to cause the one or more processors to execute the method of any of aspects 12-21.
23. A system comprising a memory storing instructions, and a processor configured to execute the instructions to perform the method of any of aspects 12-21.
24. A method for prompting a user of a medication delivery device to connect the device to a user account of the user, comprising: receiving, at a computing device, a message from the medication delivery device, the message comprising: identification information for the medication delivery device; and dose information indicative of a number of doses administered by the medication delivery device; determining, based on the identification information of the medication delivery device, whether the medication delivery device is associated with any user account; upon determining that the medication delivery device is not associated with any user account, and when the number of doses administered satisfies error message criteria, displaying an error message at the computing device indicating that the medication delivery device should be associated with the user account of the user, without preventing the user from using the medication delivery device prior to associating the medication delivery device with any user account.
25. The method of aspect 24, wherein determining whether the medication delivery device is associated with any user account comprises: determining whether the identification information is in a list of medication delivery devices connected with the user account of the user, wherein the list is stored at the computing device.
26. The method of aspect 25, further comprising, upon determining the identification information is not in the list: transmitting the identification information to a remote computing device; receiving, from the remote computing device, a second message comprising data indicative of whether the medication delivery device is associated with any user account; and analyzing the received data to determine whether the medication delivery device is associated with any user account.
27. The method of aspect 24, wherein determining whether the medication delivery device is associated with any user account comprises: transmitting the identification information to a remote computing device; receiving, from the remote computing device, a second message comprising data indicative of whether the medication delivery device is associated with any user account; and analyzing the received data to determine whether the medication delivery device is associated with any user account.
28. The method of aspect 24, wherein: the message further comprises connection data indicating whether the medication delivery device is connected to any user account; and determining whether the medication delivery device is associated with any user account comprises determining whether the connection data indicates that the medication delivery device is connected to any user account.
29. The method of aspect 28, wherein the connection data comprises a connection flag indicating whether or not the medication delivery device is connected to any account.
30. The method of aspect 29, wherein the identification information comprises a serial number associated with the medication delivery device.
31. The method of aspect 29, wherein the dose information comprises a dose event record indicative of an amount of medication that has been delivered using the medication delivery device.
32. The method of aspect 31, wherein the dose event record comprises information indicative of a dosage time.
33. The method of aspect 24, wherein the number of doses administered satisfies the error message criteria when the number of doses administered is greater than a minimum threshold.
34. The method of aspect 33, wherein the minimum threshold is zero.
35. The method of aspect 24, wherein the number of doses administered satisfies the error message criteria when the number of doses administered is greater than a minimum threshold and smaller than a maximum threshold.
36. A non-transitory computer-readable media comprising instructions that, when executed by one or more processors on a computing device, are operable to cause the one or more processors to execute the method of any of aspects 24-35.
37. A system for prompting a user of a medication delivery device to connect the device to a user account of the user, the system comprising a memory storing instructions, and a processor configured to execute the instructions to: receive, from the medication delivery device, a message comprising: identification information for the medication delivery device; and dose information indicative of a number of doses administered by the medication delivery device; determine, based on the identification information of the medication delivery device, whether the medication delivery device is associated with any user account; and upon determining that the medication delivery device is not associated with any user account, and when the number of doses administered satisfies error message criteria, displaying an error message indicating that the medication delivery device should be associated with the user account of the user, without preventing the user from using the medication delivery device prior to associating the medication delivery device with any user account.
38. The system of aspect 37, wherein determining whether the medication delivery device is associated with any user account comprises: determining whether the identification information is in a list of medication delivery devices associated with the user account of the user, wherein the list is stored in the memory.
39. The system of aspect 38, wherein the instructions are further configured to cause the processor to, upon determining the identification information is not in the list: transmit the identification information to a remote computing device; receive, from the remote computing device, a second message comprising data indicative of whether the medication delivery device is associated with any user account; and analyze the received data to determine whether the medication delivery device is associated with any user account.
40. The system of aspect 37, wherein determining whether the medication delivery device is associated with any user account comprises: transmitting the identification information to a remote computing device; receiving, from the remote computing device, a second message comprising data indicative of whether the medication delivery device is associated with any user account; and analyzing the received data to determine whether the medication delivery device is associated with any user account.
41. The system of aspect 37, wherein: the message further comprises connection data indicating whether the medication delivery device is connected to any user account; and determining whether the medication delivery device is associated with any user account comprises determining whether the connection data indicates that the medication delivery device is connected to any user account.
42. The system of aspect 41, wherein the connection data comprises a connection flag indicating whether or not the medication delivery device is connected to any account.
43. The system of aspect 37, wherein the identification information comprises a serial number associated with the medication delivery device.
44. The system of aspect 37, wherein the dose information comprises a dose event record indicative of an amount of medication that has been delivered using the medication delivery device.
45. The system of aspect 44, wherein the dose event record comprises information indicative of a dosage time.
46. The system of aspect 37, wherein the number of doses administered satisfies the error message criteria when the number of doses administered is greater than a minimum threshold.
47. The system of aspect 46, wherein the minimum threshold is zero.
48. The system of aspect 37, wherein the number of doses administered satisfies the error message criteria when the number of doses administered is greater than a minimum threshold and smaller than a maximum threshold.
49. A method for retiring a medication delivery device, comprising: accessing data associated with a medication delivery device, wherein: the medication delivery device is in a list of active medication delivery devices associated with a user account; and the medication delivery device comprises: a reservoir sized sufficiently to hold medication for a plurality of doses; and a wireless communication unit that wirelessly transmits information about the medication delivery device; determining, based on the accessed data, to retire the medication delivery device; and retiring the medication delivery device, wherein retiring the medication delivery device comprises at least one of: (a) removing the medication delivery device from the list of active medication delivery devices associated with the user account; and (b) moving an entry for the medication delivery device from the list of active medication delivery devices associated with the user account to a list of retired medication delivery devices.
50. The method of aspect 49, wherein: the accessed data comprises a time of use of the medication delivery device; and determining to retire the medication delivery device comprises determining the time of use is greater than a threshold time.
51. The method of aspect 50, wherein the time of use of the medication delivery device is determined based on a difference between a current time and one of (a) a time when the medication delivery device was first associated with the user account, and (b) a time when the medication delivery device delivered a first dose.
52. The method of aspect 50, wherein the threshold time comprises a number of days.
53. The method of aspect 49, wherein: the accessed data comprises data indicative of a battery level of the medication delivery device; and determining to retire the medication delivery device comprises determining the battery level is below a predetermined threshold.
54. The method of aspect 49, wherein: the accessed data comprises data indicative of a remaining amount of medicine of the medication delivery device; and determining to retire the medication delivery device comprises determining the remaining amount of medicine is below a predetermined threshold.
55. The method of aspect 49, wherein: the accessed data comprises data indicative of a remaining amount of medicine of the medication delivery device; and determining to retire the medication delivery device comprises determining, based on the remaining amount of medicine, the medication delivery device has no remaining medicine.
56. The method of aspect 49, wherein determining to retire the medication delivery device comprises: requesting input from a user to provide data indicating whether the medication delivery device should be retired; and determining, responsive to the received data, to retire the medication delivery device.
57. The method of aspect 49, wherein the medication delivery device further comprises: a dose adjustment mechanism that allows a user to adjust a dose amount for an injection of the medication delivery device; and one or more sensors to detect an amount of dose dispensed for each injection.
58. A non-transitory computer-readable media comprising instructions that, when executed by one or more processors on a computing device, are operable to cause the one or more processors to execute the method of any of aspects 49-57.
59. A system for retiring a medication delivery device, the system comprising a memory storing instructions, and a processor configured to execute the instructions to: access data associated with a medication delivery device, wherein: the medication delivery device is in a list of active medication delivery devices associated with a user account; and the medication delivery device comprises: a reservoir sized sufficiently to hold medication for a plurality of doses; and a wireless communication unit that wirelessly transmits information about the medication delivery device; determine, based on the accessed data, to retire the medication delivery device; and retire the medication delivery device, wherein retiring the medication delivery device comprises at least one of: removing the medication delivery device from the list of active medication delivery devices associated with the user account and moving an entry for the medication delivery device from the list of active medication delivery devices associated with the user account to a list of retired medication delivery devices.
60. The system of aspect 59, wherein: the accessed data comprises a time of use of the medication delivery device; and determining to retire the medication delivery device comprises determining the time of use is greater than a threshold time.
61. The system of aspect 60, wherein the time of use of the medication delivery device is determined based on a difference between a current time and one of (a) a time when the medication delivery device was first associated with the user account, and (b) a time when the medication delivery device delivered a first dose.
62. The system of aspect 60, wherein the threshold time comprises a number of days.
63. The system of aspect 60, wherein: the accessed data comprises data indicative of a battery level of the medication delivery device; and determining to retire the medication delivery device comprises determining the battery level is below a predetermined threshold.
64. The system of aspect 60, wherein: the accessed data comprises data indicative of a remaining amount of medicine of the medication delivery device; and determining to retire the medication delivery device comprises determining the remaining amount of medicine is below a predetermined threshold.
65. The method of aspect 60, wherein: the accessed data comprises data indicative of a remaining amount of medicine of the medication delivery device; and determining to retire the medication delivery device comprises determining, based on the remaining amount of medicine, the medication delivery device has no remaining medicine.
66. The method of aspect 60, wherein determining to retire the medication delivery device comprises: requesting input from a user to provide data indicating whether the medication delivery device should be retired; and determining, responsive to the received data, to retire the medication delivery device.
67. The method of aspect 60, wherein the medication delivery device further comprises: a dose adjustment mechanism that allows a user to adjust a dose amount for an injection of the medication delivery device; and one or more sensors to detect an amount of dose dispensed for each injection.

1. A method for presenting information from a particular medication delivery device to a user, the method comprising:
receiving a set of advertising packets, the set of advertising packets comprising at least one advertising packet from one or more medication delivery devices, the one or more medication delivery devices including the particular medication delivery device, wherein each advertising packet includes an identifier associated with a respective medication delivery device of the one or more medication delivery devices;
determining a number of unique identifiers included in the set of advertising packets to determine a number of transmitting medication delivery devices;
determining, based on the number of transmitting medication delivery devices, whether to prompt the user to provide identification information for the particular medication delivery device; and
presenting the information from the particular medication delivery device to the user.

2. The method of claim 1, further comprising:
upon determining that the number of transmitting medication delivery devices includes more than one medication delivery device, prompting the user to provide the identification information for the particular medication delivery device;
receiving the identification information; and
identifying, using the identification information, an advertising packet of the at least one advertising packet, the advertising packet having been received from the particular medication delivery device,
wherein presenting the information from the particular medication delivery device to the user comprises presenting information included in the identified advertising packet.

3. The method of claim 1, further comprising:
upon determining that the number of transmitting medication delivery devices includes more than one medication delivery device, prompting the user to provide the identification information for the particular medication delivery device;
receiving the identification information;
transmitting, based on the identification information, a query configured to provoke a response from the particular medication delivery device; and
receiving the response from the particular medication delivery device;
wherein presenting the information from the particular medication delivery device to the user comprises presenting information included in the response from the particular medication delivery device.

4. The method of claim 1, further comprising:
upon determining that the number of transmitting medication delivery devices does not include more than one medication delivery device, presenting the information from the particular medication delivery device without prompting the user to provide the identification information for the particular medication delivery device,
wherein presenting the information from the particular medication delivery device comprises presenting information included in an advertising packet of the at least one advertising packet.

5. The method of claim 1, further comprising:
upon determining that the number of transmitting medication delivery devices does not include more than one medication delivery device, transmitting, based on the identifier for each advertising packet, a query configured to provoke a response from the particular medication delivery device without prompting the user to provide the identification information for the particular medication delivery device; and
receiving the response from the particular medication delivery device;
wherein presenting the information from the particular medication delivery device to the user comprises presenting information included in the response from the particular medication delivery device.

6. The method of claim 1 wherein the information from the particular medication delivery device comprises information indicative of one or more of:
a status of the particular medication delivery device,
a time associated with a dose of medication delivered using the particular medication delivery device, or
a dosage amount associated with the dose of medication.

7. The method of claim 6, wherein the information indicative of the status of the particular medication delivery device comprises one or more of:
information indicative of a power level of the particular medication delivery device,
information indicative of an amount of medication held in the particular medication delivery device,
information indicative of a type of medication held in the particular medication delivery device,
information indicative of a time the particular medication delivery device transitioned to a connected state, or
information indicative of a time the particular medication delivery device was activated.

8. The method of claim 1, wherein prompting the user to provide the identification information comprises prompting the user to perform one or more of: scanning a barcode associated with the medication delivery device or providing input through a user interface.

9. The method of claim 1, further comprising receiving input requesting the information from the particular medication delivery device, wherein determining whether the number of transmitting medication delivery devices includes more than one medication delivery device is responsive to receiving the input.

10. A non-transitory computer-readable media comprising instructions that, when executed by one or more processors on a computing device, are operable to cause the one or more processors to:
receive a set of advertising packets, the set of advertising packets comprising at least one advertising packet from one or more medication delivery devices, the one or more medication delivery devices including the particular medication delivery device, wherein each advertising packet includes an identifier associated with a respective medication delivery device of the one or more medication delivery devices;
determine a number of unique identifiers included in the set of advertising packets to determine a number of transmitting medication delivery devices;
determine, based on the number of transmitting medication delivery devices, whether to prompt the user to provide identification information for the particular medication delivery device; and
present the information from the particular medication delivery device to the user.

11. A system for presenting information from a particular medication delivery device to a user, the system comprising:
a memory storing instructions, and
a processor configured to execute the instructions to:
receive a set of advertising packets, the set of advertising packets comprising at least one advertising packet from one or more medication delivery devices, the one or more medication delivery devices including the particular medication delivery device, wherein each advertising packet includes an identifier associated with a respective medication delivery device of the one or more medication delivery devices;
determine a number of unique identifiers included in the set of advertising packets to determine a number of transmitting medication delivery devices;
determine, based on the number of transmitting medication delivery devices, whether to prompt the user to provide identification information for the particular medication delivery device; and
present the information from the particular medication delivery device to the user.

12-58. (canceled)

59. The system of claim 11, wherein, upon determining that the number of transmitting medication delivery devices includes more than one medication delivery device, the processor is further configured to execute the instructions to:
prompt the user to provide the identification information for the particular medication delivery device;
receive the identification information; and
identify, using the identification information, an advertising packet of the at least one advertising packet, the advertising packet having been received from the particular medication delivery device,
wherein presenting the information from the particular medication delivery device to the user comprises presenting information included in the identified advertising packet.

60. The system of claim 11, wherein, upon determining that the number of transmitting medication delivery devices includes more than one medication delivery device, the processor is further configured to execute the instructions to:
receive the identification information;
transmit, based on the identification information, a query configured to provoke a response from the particular medication delivery device; and
receive the response from the particular medication delivery device;
wherein presenting the information from the particular medication delivery device to the user comprises presenting information included in the response from the particular medication delivery device.

61. The system of claim 11, wherein, upon determining that the number of transmitting medication delivery devices does not include more than one medication delivery device, the processor is further configured to execute the instructions to:
present the information from the particular medication delivery device without prompting the user to provide the identification information for the particular medication delivery device, wherein presenting the information from the particular medication delivery device comprises presenting information included in an advertising packet of the at least one advertising packet.

62. The system of claim 11, wherein, upon determining that the number of transmitting medication delivery devices does not include more than one medication delivery device, the processor is further configured to execute the instructions to:
transmit, based on the identifier for each advertising packet, a query configured to provoke a response from the particular medication delivery device without prompting the user to provide the identification information for the particular medication delivery device; and
receive the response from the particular medication delivery device;
wherein presenting the information from the particular medication delivery device to the user comprises presenting information included in the response from the particular medication delivery device.

63. The system of claim 11, wherein the information from the particular medication delivery device comprises information indicative of one or more of:
a status of the particular medication delivery device,
a time associated with a dose of medication delivered using the particular medication delivery device, or
a dosage amount associated with the dose of medication.

64. The system of claim 63, wherein the information indicative of the status of the particular medication delivery device comprises one or more of:
information indicative of a power level of the particular medication delivery device,
information indicative of an amount of medication held in the particular medication delivery device,
information indicative of a type of medication held in the particular medication delivery device,
information indicative of a time the particular medication delivery device transitioned to a connected state, or
information indicative of a time the particular medication delivery device was activated.

65. The system of claim 11, wherein prompting the user to provide the identification information comprises prompting the user to perform one or more of: scanning a barcode associated with the medication delivery device or providing input through a user interface.

66. The system of claim 11, wherein the processor is further configured to execute the instructions to receive input requesting the information from the particular medication delivery device, wherein determining whether the number of transmitting medication delivery devices includes more than one medication delivery device is responsive to receiving the input.
